(19) United States
(12) Patent Application Publication
(10) Pub. No.: US 2023/0248338 A1
(43) Pub. Date: Aug. 10, 2023

(54) ANTERIOR NARES SWAB AND USES THEREOF

(71) Applicant: PRESIDENT AND FELLOWS OF HARVARD COLLEGE, Cambridge, MA (US)

(72) Inventors: Michael SPRINGER, Cambridge, MA (US); Richard NOVAK, Cambridge, MA (US)

(73) Assignee: PRESIDENT AND FELLOWS OF HARVARD COLLEGE, Cambridge, MA (US)

(21) Appl. No.: 18/005,276

(22) PCT Filed: Jul. 13, 2021
(86) PCT No.: PCT/US2021/041429

§ 371 Date: Jan. 12, 2023

Related U.S. Application Data
(60) Provisional application No. 63/085,571, filed on Sep. 30, 2020.
(60) Provisional application No. 63/051,263, filed on Jul. 13, 2020.

(51) Int. Cl. A61B 10/00; B01L 3/00

(52) U.S. Cl. CPC A61B 10/0051; B01L 3/50825; B01L 2300/042

(57) ABSTRACT

The technology described herein is directed to an anterior nares swab that is automation compatible. In one aspect, the swab comprises a cap, a threaded portion, a neck, and a sample collection head. The cap can be integrally and/or monolithically formed with any one or more of the threaded portion, the neck, and the sample collection head; or can be removably coupled to any one or more of the threaded portion, the neck, and the sample collection head. In additional aspects, described herein are kits comprising said swabs and methods of using said swabs.

### CROSS-REFERENCE TO RELATED APPLICATIONS

This application claims priority to and the benefit of under 35 U.S.C. § 119(e) U.S. Provisional Patent Application No. 63/051,263 filed Jul. 13, 2020, and U.S. Provisional Patent Application No. 63/085,571 filed Sep. 30, 2020, each of which is hereby incorporated by reference herein in its entirety

### GOVERNMENT SUPPORT

This invention was made with government support under Grant No. D18AC00006 awarded by the U.S. Department of Defense, Defense Advanced Research Projects Agency (DARPA). The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jul. 13, 2021, is named 002806-098030WOPT_SL.txt and is 1,084 bytes in size.

### TECHNICAL FIELD

The technology described herein relates to an anterior nares swab and uses thereof.

### BACKGROUND

One of the key limitations to high throughput diagnostic test, e.g. COVID-19 viral assay, is the time it takes to remove the swab from the sample tube and then transfer the sample to the assay device. This typically involves laboratory personnel: taking a single sample into a biosafety level 2 (BSL2) space; taking out the swab; transferring the sample; sealing the tube; and then repeating. People have been trying to design ways for swabs to be automation compatible, but there is currently no solution that address the swab in tube problem besides manually removing the tube and/or pipetting out the fluid into a new receptacle compatible with automation. There is a great need for sample collection swabs that reduce the processing time for a sample and are automation compatible.

### SUMMARY

The technology described herein is directed to an anterior nares swab that is automation compatible. In one aspect, the swab comprises a cap, a threaded portion, a neck, and a sample collection head. The swab as described herein comprises at least one of the following features: (1) saves full-time equivalent (FTE) hours; (2) saves space in a Clinical Laboratory Improvement Amendments (CLIA) lab; (3) allows high throughput automation of swab removal; (4) speeds the connection of sample accession to diagnostic result; (5) single shot injection molded process which can allow for cheap and easy manufacturing; (6) head design (e.g., comprising annular rings as described further herein) reduces likelihood of dripping or other cross contamination; (7) compatible with dry or wet transport and self-swabbing at home or at test sites; (8) reduced material consumption due to small size/mass and avoiding need for additional plasticware; (9) cap is used as a handle and prevent risk to patients from over-insertion of swab in the nose; (10) no need to break swab for collection, which minimizes contamination and infection risk; (11) viral stability on swab (e.g., for at least 72 hours); (12) viral stability on swab at high temperature (e.g., 42° C.); (13) viral stability on swab in dry conditions; or (14) ability to elute the sample from the swab in a low volume of liquid (e.g., 200 uL). In additional aspects, described herein are kits comprising said swabs and methods of using said swabs.

In one aspect described herein, a swab comprises a cap, a neck, and a sample collection head formed from a non-flocked material.

In some embodiments of any of the aspects, the swab further comprises a threaded portion.

In some embodiments of any of the aspects, the cap is removably coupled to the threaded portion, the neck, the sample collection head, or any combination thereof.

In some embodiments of any of the aspects, the cap comprises a hollow cylinder with at least one internal groove or at least one internal ridge.

In some embodiments of any of the aspects, the cap can interface with an automated device.

In some embodiments of any of the aspects, the automated device is a tube capper and decapper machine.

In some embodiments of any of the aspects, wherein the threaded portion of the swab is configured to interface with a container tube.

In some embodiments of any of the aspects, the threaded portion of the swab is configured to interface with a threaded portion of the container tube.

In some embodiments of any of the aspects, the head comprises a plurality of spaced apart annular rings, a spiral axis groove, a bulb, a stippled surface, a roughened surface, a textured surface, or any combination thereof.

In some embodiments of any of the aspects, the swab is injection molded.

In some embodiments of any of the aspects, the threaded portion, the neck, and the sample collection head are fabricated as a unitary item via injection molding, and the unitary item is then adhered to the cap.

In some embodiments of any of the aspects, the cap is aligned off-axis relative to the sample collection head.

In some embodiments of any of the aspects, the sample collection head is aligned on a first axis, and the cap is aligned on a second axis, the first axis and the second axis being two distinct axes.

In some embodiments of any of the aspects, the first axis and the second axis are parallel to each other and spaced apart from each other.

In some embodiments of any of the aspects, the first axis and the second axis are not coaxial.

In some embodiments of any of the aspects, the neck is aligned on the first axis with the sample collection head, and wherein the threaded portion is aligned on the second axis with the cap.

In some embodiments of any of the aspects, the cap is configured to be grasped by a user.

In some embodiments of any of the aspects, the swab further comprises a handle portion coupled to the cap.

In some embodiments of any of the aspects, the handle portion extends away from the cap such that the cap is positioned between the handle portion and the neck.

In some embodiments of any of the aspects, a width of a distal end of the handle portion adjacent to the cap is generally equal to a width of the cap.

In some embodiments of any of the aspects, the handle portion has a tapered shape, the handle portion including a distal end having a first diameter and a proximal end having a second diameter.

In some embodiments of any of the aspects, the first diameter is less than the second diameter.

In some embodiments of any of the aspects, the handle portion is removably coupled to the cap.

In some embodiments of any of the aspects, the handle portion is configured to detach from the cap in response to the cap being coupled to a container tube.

In some embodiments of any of the aspects, the handle portion is configured to detach from the cap in response to the cap being coupled to the container tube with a correct amount of force or tightness.

In some embodiments of any of the aspects, the detaching of the handle portion indicates that the cap is sufficiently coupled to the container tube.

In some embodiments of any of the aspects, the handle portion is configured to detach from cap in response to application of an external force.

In some embodiments of any of the aspects, the swab further comprises a guard positioned at an end of the handle portion adjacent to the cap.

In some embodiments of any of the aspects, the guard has a circular shape extending in a plane, and wherein the handle portion extends normal to a plane of the guard.

In some embodiments of any of the aspects, the cap, the threaded portion, the neck, and the sample collection head comprise the same material.

In some embodiments of any of the aspects, the material is a flexible polymer.

In some embodiments of any of the aspects, the material is polypropylene.

In some embodiments of any of the aspects, the material is biodegradable.

In some embodiments of any of the aspects, the material is water-soluble.

In some embodiments of any of the aspects, the material is hydrophobic.

In some embodiments of any of the aspects, the material is polyvinyl alcohol (PVA).

In some embodiments of any of the aspects, the material is foam or a porous material.

In some embodiments of any of the aspects, the head comprises a fibrous coating.

In some embodiments of any of the aspects, the sample collection head comprises a first material, and the remainder of the swab comprises a second material.

In some embodiments of any of the aspects, the sample collection head comprises a water-soluble or biodegradable material and the remainder of the swab comprises a flexible polymer.

In some embodiments of any of the aspects, the sample collection head comprises PVA and the remainder of the swab comprises polypropylene.

In some embodiments of any of the aspects, the neck tapers from a maximum diameter towards the cap to a minimum diameter towards the head.

In some embodiments of any of the aspects, the swab has a length that is at most 100 mm.

In some embodiments of any of the aspects, the swab has a length that is at most 50 mm.

In some embodiments of any of the aspects, the swab is in combination with a container tube.

In one aspect described herein is a kit comprising the swab of any of the embodiments.

In some embodiments of any of the aspects, the kit further comprises a container tube.

In one aspect described herein, a method of collecting a sample comprising contacting a sample with the swab of any one of embodiments.

In some embodiments of any of the aspects, the sample is an anterior nares epithelial surface of a subject.

In some embodiments of any of the aspects, the subject is infected with or suspected to be infected with a respiratory infection.

In some embodiments of any of the aspects, after the contacting step, the swab is deposited into a container tube.

In some embodiments of any of the aspects, after the swab is deposited into a container tube, the sample is processed using at least one automated device.

In some embodiments of any of the aspects, the automated device is selected from the group consisting of: a tube capper and decapper machine, a liquid handling machine, and a shaker.

In some embodiments of any of the aspects, the swab does not inhibit or reduce a downstream application.

In one aspect described herein, an automated method of processing a swab comprises receiving a swab of any of the embodiments, wherein the swab has been contacted with a sample and deposited into a container tube; removing at least a portion of the sample from the sample collection head using a tube capper and decapper machine, a liquid handling machine, and a shaker; and processing the at least a portion of the sample using a downstream application.

In some embodiments of any of the aspects, after receiving the swab, a barcode and/or label on the swab and/or collection tube is detected using a barcode scanning machine.

In some embodiments of any of the aspects, removing at least a portion of the sample from the sample collection head comprises removing the swab from the sample collection tube using the tube capper and decapper machine; adding a solution to the sample collection tube using the liquid handling machine; replacing the swab into the sample collection tube using the tube capper and decapper machine; shaking the solution in the tube in a shaker in order to remove at least a portion of the sample from the sample collection head of the swab; removing the swab from the sample collection tube and solution using the tube capper and decapper machine; and removing a portion of the solution from the sample collection tube using the liquid handling machine for the downstream application.

In some embodiments of any of the aspects, the solution is saline.

In some embodiments of any of the aspects, the step of removing at least a portion of the sample from the sample collection head is conducted in about 6 minutes.

In some embodiments of any of the aspects, the downstream application includes a nucleic acid extraction step.

In some embodiments of any of the aspects, the downstream application includes RT-qPCR.

### BRIEF DESCRIPTION OF THE DRAWINGS

FIG. 1 is a perspective view of a one shot injection molded swab, according to aspects of the present disclosure.

FIG. 2A is an image of the swab of FIG. 1 inside a barcoded collection tube, and a pen-like device that simulates how a robot head engages the cap, according to aspects of the present disclosure.

FIG. 2B is an image of the swab of FIG. 1 showing compatibility with a 1.0 mL tube, according to aspects of the present disclosure.

FIG. 2C is an image of the swab of FIG. 1 in standard matrix tube demonstrating the seal between the cap and the tube, according to aspects of the present disclosure.

FIG. 3 is an engineering drawing showing exemplary dimensions of the swab of FIG. 1, according to aspects of the present disclosure.

FIG. 4 is a bar graph showing RT-qPCR for Human glyceraldehyde 3-phosphate dehydrogenase (GAPDH)mRNA from a series of 11 different AN swabs, according to aspects of the present disclosure. Swabs 3 is the swab as described herein (see e.g., Table 2). The other numbers are other swabs that have been approved for use. The swabs as described herein perform comparable for capture and release whether by release by vortexing or spinning.

FIG. 5 is a schematic showing an exemplary workflow using cap-integrated swabs, according to aspects of the present disclosure.

FIG. 6 shows a swab with a cap aligned off-axis relative to a sample collection head, according to aspects of the present disclosure.

FIG. 7A and FIG. 7B show a swab with a handle portion, according to aspects of the present disclosure.

FIG. 8A and FIG. 8B show a swab with a handle portion and a guard, according to aspects of the present disclosure.

FIG. 9A and FIG. 9B show a swab with a cap including internal features that aid in allowing an automated device to interface with the cap.

FIGS. 10A-10D show a series of images showing an exemplary swabs and workflow, according to aspects of the present disclosure. FIG. 10A shows a custom injection molded AN swab that can be produced at large scale and is compatible with SBS 24-, 48-, and/or 96-well format automation. FIG. 10B shows a sample nose as scale bar for swab. FIG. 10C shows a 96-well rack of swabs and tubes. FIG. 10D shows a 2D barcode on bottom of tubes. All 96 barcodes can be read rapidly in one shot by a scanner.

FIG. 11A-11B shows a 96-well format automation and accession compatible AN swab design, according to aspects of the present disclosure. FIG. 11A shows an image of a custom injection molded AN swab that can be produced at large scale and is compatible with 96-well format automation. A sample tube compatible with the RHINOstic™ swab is shown with barcodes on the side and bottom. The RHINOstic™ swab is 4.9 cm long with a collection head length of 1.6 cm. 1 cm scale bar shown for reference. FIG. 11B shows an image of a 96-well rack of swabs and tubes with 2D matrix codes printed on the bottom of the tubes, allows for rapid accessioning.

FIG. 12A-12E shows a comparison of swab performance, according to aspects of the present disclosure. FIG. 12A shows an image of AN swabs tested in this study, from left to right: RHINOstic™, Proctor & Gamble™ (P&G) blue, Wyss Institute™ flocked prototype, Puritan™ hydraflock, Puritan™ foam, Puritan™ polyester, US Cotton™, and Microbrush®. 1 cm scale bar shown for reference. FIG. 12B shows a schematic of swab experiments performed in FIG. 12C-12D. FIG. 12B scheme I: SARS-CoV-2 negative volunteer self-collected nasal matrix on a swab. FIG. 12B scheme II: unused swab, without nasal matrix, was either treated with packaged synthetic SARS-CoV-2 virus or left untreated (clean, unused swab). FIG. 12B scheme III: SARS-CoV-2 negative volunteer self-collected nasal matrix on a swab which was then treated with packaged synthetic SARS-CoV-2 or SARS-CoV-2 clinical sample (see e.g., Methods). All samples were eluted in PBS and used as direct input to RT-qPCR assays. Images created with BioRender.com. FIG. 12C is a bar graph showing RT-qPCR quantitation of human GAPDH mRNA from used swabs containing nasal matrix (pink bars) or matched unused swabs (grey bars). FIG. 12D is a bar graph showing RT-qPCR quantitation of the SARS-CoV-2 N gene from packaged synthetic virus applied to clean, unused swabs. The grey bar is the negative control, PBS input into RT-qPCR. The pink line is a guideline for complete recovery based on the positive control. FIG. 12E is a bar graph showing RT-qPCR quantitation of SARS-CoV-2 N gene from swabs in the presence of nasal matrix spiked with a lower (˜140 copies/μL, pink bars) or higher (˜1600 copies/μL, green bars) titer clinical sample. The grey bar is the negative control, PBS, and the positive controls are the lower or higher titer clinical samples directly input to RT-qPCR. RT-qPCR data in FIG. 12C-12E all show technical replicates of at least 3 biological experiments.

FIG. 13A-13E shows the stability of SARS-CoV-2 on swabs in the presence of nasal matrix, according to aspects of the present disclosure. FIG. 13A shows a schematic of the experimental workflow in FIG. 13B-13E. SARS-CoV-2 clinical sample was applied to unused swabs or self-collected AN swabs, with nasal matrix, (see e.g., Methods) and left dry or wet at 25° C., for up to 72 hours. All samples were quantified by direct input of eluent into RT-qPCR. Images created with BioRender.com. FIG. 13B-13C are a series of bar graphs showing the stability of SARS-CoV-2 on RHINOstic™ swabs with nasal matrix left dry or wet at 25° C. or dry at 42° C. analyzed over the course of 72 hours by RT-qPCR for the SARS-CoV-2 N gene (see e.g., FIG. 13B) or GAPDH (see e.g., FIG. 13C). FIG. 13D-13E are a series of bar graphs showing the stability of SARS-CoV-2 on Puritan™ foam swabs with nasal matrix left dry or wet at 25° C. or dry at 42° C. was analyzed over the course of 72 hours by RT-qPCR for the SARS-CoV-2 N gene (see e.g., FIG. 13D) or GAPDH (see e.g., FIG. 13E). Data points in FIG. 13B-13E are technical replicates of 2 biological replicates. The positive control in FIG. 13B-13E is the SARS-CoV-2 clinical sample directly added to PBS at time 0. The negative control is an unused RHINOstic™ (see e.g., FIG. 13B-13C) or Puritan™ foam (see e.g., FIG. 13D-13E) swab in PBS.

FIG. 14A-14F shows the elution of viral particles, according to aspects of the present disclosure. FIG. 14A is a bar graph showing RT-qPCR quantitation of the release of synthetic SARS-CoV-2 from unused RHINOstic™ or Puritan™ foam swabs into PBS by either vortexing on high or manually spinning the swab in the elution tube for 10 seconds. The positive control is 10 copies of packaged synthetic SARS-CoV-2 virus and the negative control is PBS. FIG. 14B is a bar graph showing RT-qPCR quantitation of the release of GAPDH from self-collected SARS-CoV-2 negative volunteer with RHINOstic™ or Puritan™ foam swabs by vortexing on high or manually spinning the swabs in the elution tube for 10 seconds. The positive control is 1.35e5 molecules of total HeLa RNA and the negative control is PBS. FIG. 14C is a bar graph showing RT-qPCR quantification of GAPDH from used RHINOstic™ or Puritan™ foam contrived swab samples used in FIG. 12E. RT-qPCR data in FIG. 14A-14C are technical replicates of at least 3 biological experiments. FIG. 14D is a dot plot showing quantification of synthetic full genome SARS-CoV-2 RNA by N gene RT-qPCR. The standard curve has an R\hat{ }2 of 0.83 and the line of best fit is y=−3.829x+36.15. FIG. 14E is a scatterplot plot showing technical replicates, Ct1 and Ct2 from FIG. 12C and FIG. 14C were plotted against each other for the RHINOstic™ and Puritan™ foam data points. The R2 values for the RHINOstic™ and Puritan™ foam swabs were 0.9791 and 0.9891, respectively. FIG. 14F is a scatterplot plot showing technical replicates, Ct1 and Ct2, from FIG. 12D and FIG. 12E comparing the RHINOstic™ and Puritan™ foam swabs are plotted against each other. The R2 values were 0.9482 and 0.8488 respectively for the RHINOstic™ and Puritan™ foam swabs.

FIG. 15A-15F shows the stability of human cells on swabs with nasal matrix, according to aspects of the present disclosure. FIG. 15A-15B are a series of bar graphs; all RT-qPCR time course data from each target in FIG. 13B-13E were averaged and compared to the unused swab. Data is labeled with the average Ct and standard deviation. FIG. 15C-15D are a series of bar graphs; SARS-CoV-2 negative volunteers self-swabbed with RHINOstic™, Puritan™ foam, and US Cotton™ swabs (see e.g., Supplemental Methods) at each time point and left them dry or in 1 mL of PBS at 25° C. (similar to the schematic in FIG. 13A). All dry samples were eluted in PBS at time 0 and used as direct input into an RT-qPCR assay for GAPDH mRNA detection. Data are technical replicates of biological duplicates. Time 0 data is the same in FIG. 15A and FIG. 15B and is replotted for clarity. Negative controls are unused swabs put directly into PBS at time 0. FIG. 15E-15F are a series of scatterplots; technical replicate 1, Ct1, was plotted against technical replicate 2, Ct2, for GAPDH (see e.g., FIG. 15E) and N gene (see e.g., FIG. 15F) data generated in the stability time course experiment plotted in FIG. 13. Res of the GAPDH data for the RHINOstic™ and Puritan™ foam swabs were 0.7734 and 0.6527, respectively. The R2 values for the RHINOstic™ and Puritan™ foam swab N-gene data was 0.5733 and 0.2827, respectively.

### DETAILED DESCRIPTION

The technology described herein is directed to an anterior nares swab that is automation compatible. In one aspect, the swab comprises a cap, a threaded portion, a neck, and a sample collection head. The swab as described herein facilitates at least one of the following advantages: (1) saves full-time equivalent (FTE) hours; (2) saves space in a Clinical Laboratory Improvement Amendments (CLIA) lab; (3) allows high throughput automation of swab removal; (4) speeds the connection of sample accession to sample; (5) single shot injection molded process which can allow for cheap and easy manufacturing; (6) head design (e.g., comprising annular rings as described further herein) reduces likelihood of dripping or other cross contamination; (7) compatible with dry or wet transport and self-swabbing at home or at test sites; (8) reduced material consumption due to small size/mass and avoiding need for additional plasticware; (9) cap is used as a handle and prevent risk to patients from over-insertion of swab in the nose; (10) no need to break swab for collection, which minimizes contamination and infection risk; (11) viral stability on swab (e.g., for at least 72 hours); (12) viral stability on swab at high temperature (e.g., 42° C.); (13) viral stability on swab in dry conditions; or (14) ability to elute the sample from the swab in a low volume of liquid (e.g., 200 uL). In additional aspects, described herein are kits comprising said swabs and methods of using said swabs.

### Swab

Described herein is a swab for sample collection. In one aspect, the swab comprises a sample collection head. In some embodiments, the swab further comprises a neck. In some embodiments, the swab further comprises a threaded portion. In some embodiments, the swab further comprises a cap. In some embodiments, the swab is in combination with a container tube. Any combination of the foregoing is contemplated herein. Exemplary combinations are shown in Table 1 below.

TABLE 1
Exemplary Swabs (an “X” indicates that the swab
comprises the indicated component; tube indicates the
container tube with which the swab can be in combination)

| | | Threaded | | |
|---|---|---|---|---|
| Head | Neck | Portion | Cap | Tube |
| X | | | | |
| X | X | | | |
| X | | X | | |
| X | X | X | | |
| X | | | X | |
| X | X | | X | |
| X | | X | X | |
| X | X | X | X | |
| X | | | | X |
| X | X | | | X |
| X | | X | | X |
| X | X | X | | X |
| X | | | X | X |
| X | X | | X | X |
| X | | X | X | X |
| X | X | X | X | X |

The components of the swab can be in any order. In some embodiments of any of the aspects, the swab comprises in the following order: head-neck-threaded portion-cap, with optional components inserted into this order. Non limiting examples of ordered components of the swab include: head-neck-threaded portion; head-neck-cap; head-threaded portion-cap; head-cap; head-neck-cap.

In some embodiments, the components of the swab are directly or indirectly connected to each other. In some embodiments, the components of the swab are aligned according to the same central axis (i.e., share the same cross-sectional midpoint). In some embodiments, one or more of the components of the swab are aligned on separate axes. For example, the head and/or the neck of the swab can be aligned on an axis separate from the axis on which the cap is aligned. This off-axis alignment can facilitate better elution of the sample from the head of the swab when using an orbital shaker or other actuation that moves liquid in the tube. Due to the off-axis alignment, the shear on the head of the swab is increased, and thus faster elution toward the tube walls can be achieved.

In some embodiments, the length of the swab (e.g., from “distal” end, which is used herein to refer to the head end, to the “proximal” end, which is used herein to refer to the non-head end, such as the cap end) is at least 70 mm. In some embodiments, the length of the swab is about 4.9 cm (49 mm). In some embodiments, the length of the swab is about 42 mm. In some embodiments, the length of the swab is about 73 mm. In some embodiments, the length of the swab is about 75 mm. In some embodiments, the length of the swab is about 82 mm. As a non-limiting example, an anterior nares swab is a sufficient length (e.g., about 75 mm) to reach the anterior nares epithelial surface of the subject. As another non-limiting example, an anterior nares swab is approximately the length of a portion of an individual's finger inserted into their nasal cavity. In some embodiments, the length of the swab is about 20 mm to 100 mm. In some embodiments, the length of the swab is at least 20 mm, at least 25 mm, at least 30 mm, at least 35 mm, at least 40 mm, at least 45 mm, at least 50 mm, at least 55 mm, at least 60 mm, at least 65 mm, at least 70 mm, at least 75 mm, at least 80 mm, at least 85 mm, at least 90 mm, at least 95 mm, or at least 100 mm.

In some embodiments, the length of the swab is at most 100 mm. In some embodiments, the length of the swab is at most 20 mm, at most 25 mm, at most 30 mm, at most 35 mm, at most 40 mm, at most 42 mm, at most 45 mm, at most 49 mm, at most 50 mm, at most 55 mm, at most 60 mm, at most 65 mm, at most 70 mm, at most 73 mm, at most 75 mm, at most 80 mm, at most 82 mm, at most 85 mm, at most 90 mm, at most 95 mm, at most 100 mm, at most 105 mm, at most 110 mm, at most 115 mm, at most 120 mm, at most 125 mm, or at most 130 mm.

In some embodiments, the length of the swab is in a range from 1 mm to 100 mm, in a range from 5 mm to 95 mm, in a range from 10 mm to 90 mm, in a range from 15 mm to 85 mm, in a range from 20 mm to 80 mm, in a range from 25 mm to 75 mm, in a range from 30 mm to 70 mm, in a range from 35 mm to 65 mm, in a range from 40 mm to 60 mm, or in a range from 45 mm to 55 mm.

In some embodiments, the swab is in combination with a container tube (see e.g., FIG. 2). In some embodiments, the swab is inserted into the container tube. In some embodiments, the container tube contains sample transport media. In some embodiments, the container tube can be constructed from a transparent material. In some embodiments, the container tube has a length that is the same as the total length of the swab. In some embodiments, the container tube has a length that is less than the total length of the swab. In some embodiments, the container tube has a length that is greater than the total length of the swab. In some embodiments, the container tube has an internal diameter that is greater than the maximum diameter of the swab. In some embodiments, the container tube has an internal diameter that is the same as the maximum diameter of the swab (e.g., the maximum diameter of the cap). In some embodiments, the container tube comprises a threaded portion. As used herein, e.g., in reference to the swab and/or collection tube, the term “threaded portion” refers to a cylindrical portion comprising raised helical thread(s). In some embodiments, the threaded portion of the container tube comprises 1, 2, 3, 4, 5, or more threads, which can be continuous or discontinuous. In some embodiments, the thread(s) wraps clockwise or counterclockwise around the container tube, e.g., when viewed from the open end of the tube. In some embodiments, the threaded portion of the container tube comprises a geometry that interfaces with the geometry of the threaded portion of the swab. In some embodiments, the container tube comprises an internally threaded portion that interfaces with an externally threaded portion of the swab. In some embodiments, the container tube comprises an externally threaded portion that interfaces with an internally threaded portion of the swab. In some embodiments, the container tube comprises internal grooves or internal ridges (also known as flanges). In some embodiments, the container tube comprises an internal or external geometric feature to permit snapping, holding in place (e.g., a bayonet mount), and/or sealing the swab and biological sample within the container tube, and the swab comprises the corresponding comprises the corresponding geometry to interface with the container tube. In some embodiments, the container tube is compatible for use with an automated device. In some embodiments, the container tube is compatible with the Society for Biomedical Sciences (SBS) 24-well format, the SBS 48-well format, the SBS 96-well format, or any combination thereof. In some embodiments, the container tube is any tube in a range from 0.1 mL to 20 mL, 0.5 mL to 15 mL, 1 mL to 10 mL, or 3 mL to 8 mL. In some embodiments, the container is a 8-mL tube. In some embodiments, the container is a 5-mL tube. In some embodiments, the container tube is a 1-mL tube. In some embodiments, the container tube is a 0.5-mL tube. In some embodiments, the length of the swab excluding the cap (e.g., the head, neck, and/or threaded portion) is less than the length of the collection tube. In some embodiments, the length of the swab excluding the cap (e.g., the head, neck, and/or threaded portion) is about 75 mm. In some embodiments, the length of the swab excluding the cap (e.g., the head, neck, and/or threaded portion) is about 20 mm to 100 mm.

In some embodiments, the swab comprises a barcode or label. In some embodiments, the barcode or label can be located on any component of the swab, e.g., the sample collection head, the neck, the threaded portion, the cap, or the container tube. In some embodiments, the barcode or label is located on the cap. In some embodiments, the barcode or label is located on the bottom of the collection tube. In some embodiments, the barcode or label is located on the side of the collection tube. In some embodiments, a barcode or label is located on multiple locations on the swab and/or collection tube, and/or located on both the swab and the collection tube, which can be the same or different barcode or label. In some embodiments, the barcode is a 1D or 2D barcode. In some embodiments, the barcode or label is laser-etched or printed. In some embodiments, the barcode or label is unique to each sample and permits identification of the sample.

### Sample Collection Head

In one aspect, the swab comprises a sample collection head. As used herein, the term “sample collection head” (or simply “head”) refers to the distal end of the swab, e.g., that is contacted with a sample to be collected; as described herein, at least a portion of the sample (e.g., mucus, cells, and microorganisms) is collected in the head of the swab, which can be used for downstream application. The sample collection head can comprise any configuration that is sufficient to collect a sample from the anterior nares (e.g., the nostrils). Non-limiting examples of sample collection heads include: a bristled head, a non-bristled head, a flocked head, a non-flocked head, and the like. In some embodiments, the sample collection head consists of a cylindrical rod with a rounded bulb at the distal end of the head.

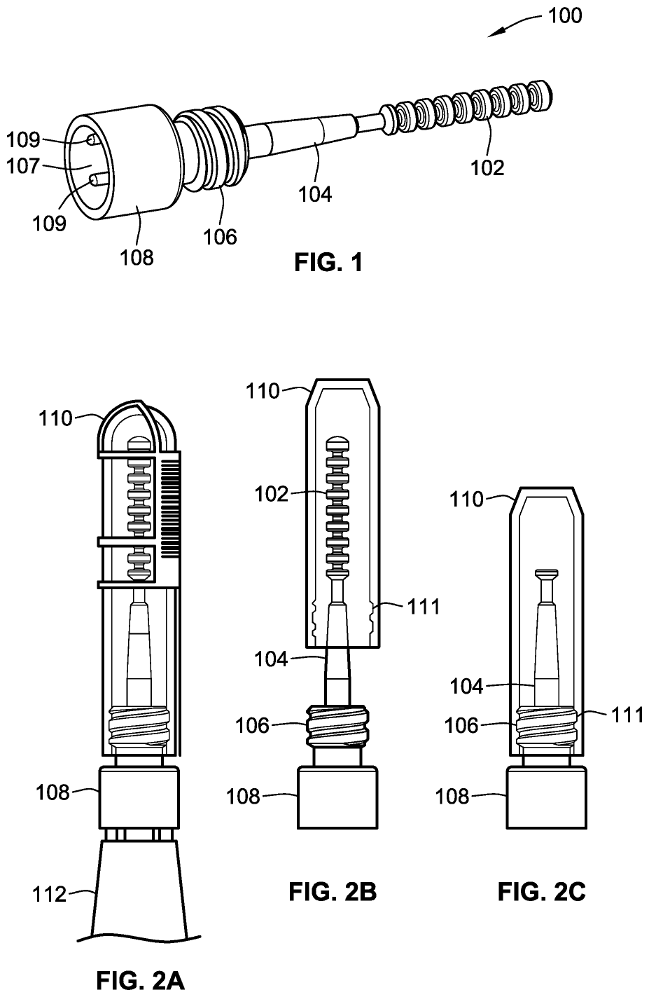

In some embodiments, the sample collection head comprises a plurality of spaced annular rings (see e.g., FIG. 1). As used herein, the term “annular ring” or “ring” refers to a projection that has a greater diameter than the diameter of an axial shaft of the collection head. As used herein, the term “axial shaft” refers to sections that connect or “run through” the spaced rings; the axial shaft can be continuous with the neck and/or cap of the swab. In some embodiments, the plurality of rings comprises 2, 3, 4, 5, 6, 7, 8, 9, 10, 11, 12, 13, 14, 15, 16, 17, 18, 19, 20, 21, 22, 23, 24, 25, or more rings. In some embodiments, the plurality of rings comprises 10 rings.

In some embodiments, the cross-section of the ring is a circle, a semicircle, a truncated circle, or a circle with one or more flat sides. In some embodiments, the cross-section of the ring is circular. In some embodiments, the ring has a polygonal cross section, e.g., a cross-section in the shape of a triangle, a square, a quadrilateral, a trapezoid, a pentagon, a hexagon, or a polygon with at least 7, 8, 9, 10, 11, 12, 13, 14, 15, 16, 17, 18, 19, 20 or more sides. In some embodiments, at least one side of the cross-section of the ring comprises a convex and/or concave curve. In some embodiments, the cross section of the ring is a rotationally symmetric shape. In some embodiments, the cross section of the ring is an asymmetric shape. In some embodiments, the ring cross-section is the same for the plurality of rings. In some embodiments, the ring cross-section is different for at least one ring in the plurality of rings; the head can comprise any combination of different (e.g., at least 2, at least 3, at least 4, at least 5) ring cross-sections.

In some embodiments, the cross-section of the axial shaft is a circle, a semicircle, a truncated circle, or a circle with one or more flat sides. In some embodiments, the cross-section of the axial shaft is circular. In some embodiments, the axial shaft comprises a cylindrical rod. In some embodiments, the axial shaft has a polygonal cross section, e.g., a cross-section in the shape of a triangle, a square, a quadrilateral, a trapezoid, a pentagon, a hexagon, or a polygon with at least 7, 8, 9, 10, 11, 12, 13, 14, 15, 16, 17, 18, 19, 20 or more sides. In some embodiments, at least one side of the cross-section of the axial shaft comprises a convex and/or concave curve. In some embodiments, the cross section of the axial shaft is a rotationally symmetric shape. In some embodiments, the cross section of the axial shaft is an asymmetric shape. In some embodiments, the axial shaft cross-section is the same for the entirety of the axial shaft. In some embodiments, the axial shaft cross-section is different for at least one portion of the axial shaft; the axial shaft can comprise any combination of different (e.g., at least 2, at least 3, at least 4, at least 5) axial shaft cross-sections.

In some embodiments, the plurality of rings is spaced apart, i.e., exposing the axial shaft. As used herein, ring spacing refers to the distance between the end of one ring to the beginning of the next ring. In some embodiments, the plurality of rings is spaced 0.1 mm-3.0 mm. In some embodiments, the plurality of rings is spaced 0.5 mm-2.0 mm. In some embodiments, the plurality of rings is spaced 0.75 mm. In some embodiments, the plurality of rings is spaced at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm. In some embodiments, the spacing between each sequential pair of rings is the same for all pairs in the head. In some embodiments, the spacing between each sequential pair of rings is different for at least one of the pairs in the head; the head can comprise any combination of different (e.g., at least 2, at least 3, at least 4, at least 5) ring spacing distances.

In some embodiments, the plurality of rings have a thickness of 0.1 mm-3.0 mm. As used herein, ring thickness refers to the distance from the beginning of a ring to the end of that same ring. In some embodiments, the plurality of rings have a thickness of 1.0 mm. In some embodiments, the plurality of rings have a thickness of 0.5 mm-2.0 mm. In some embodiments, the plurality of rings have a thickness of 0.75 mm. In some embodiments, the plurality of rings have a thickness of at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm. In some embodiments, the ring thickness is the same for the plurality of rings. In some embodiments, at least one head is a different thickness than another ring in the plurality of rings; the head can comprise any combination of different (e.g., at least 2, at least 3, at least 4, at least 5) ring thicknesses.

In some embodiments, the plurality of rings have a thickness of at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, or at most 3 mm.

In some embodiments, the plurality of rings have a diameter of 1.0 mm-4.0 mm. As used herein, the term “diameter” refers to the distance of a straight line passing through the axial center of a circular cross section (e.g., taken perpendicular to the axial shaft). In some embodiments, the plurality of rings have a diameter of 2.5 mm. In some embodiments, the plurality of rings have a diameter of 1.0 mm. In some embodiments, the plurality of rings have a diameter of at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm. In some embodiments, the ring diameter is the same for the plurality of rings. In some embodiments, at least one ring is a different diameter than another ring in the plurality of rings; the head can comprise any combination of different (e.g., at least 2, at least 3, at least 4, at least 5) ring diameters.

In some embodiments, the plurality of rings have a diameter that is less than the narrowest section of the nasal cavity (e.g., less than 4 mm). In some embodiments, the plurality of rings have a diameter of at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, or at most 4 mm.

In some embodiments, the axial shaft has a diameter of 0.5 mm-4.0 mm. By definition, the diameter of the axial shaft is less than the diameter of the proximate rings. In some embodiments, the axial shaft has a diameter of 1.2 mm. In some embodiments, the axial shaft has a diameter of at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm. In some embodiments, the axial shaft diameter is constant throughout the head. In some embodiments, the axial shaft diameter is the same diameter as the diameter of the distal region of the neck. In some embodiments, at least one portion of the axial shaft is a different diameter than portion of the axial shaft; the axial shaft can comprise any combination of different (e.g., at least 2, at least 3, at least 4, at least 5) diameters.

In some embodiments, the axial shaft has a diameter of at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, or at most 3.95 mm.

As described herein, the term “annular ring” or “ring” refers to a circular projection that has a greater diameter than the diameter of an axial shaft of the collection head. Accordingly, the height of a ring (e.g., from the axial shaft to the widest diameter of the ring) can be calculated as half of the difference between the diameter of the ring and the diameter of the axial shaft. In some embodiments, the plurality of rings have a height of 0.5 mm-1.75 mm. In some embodiments, the plurality of rings have a height of 0.65 mm (e.g., 0.5*(2.5-1.2)). In some embodiments, the plurality of rings have a height of at least 0.5 mm, at least 0.51 mm, at least 0.52 mm, at least 0.53 mm, at least 0.54 mm, at least 0.55 mm, at least 0.56 mm, at least 0.57 mm, at least 0.58 mm, at least 0.59 mm, at least 0.6 mm, at least 0.61 mm, at least 0.62 mm, at least 0.63 mm, at least 0.64 mm, at least 0.65 mm, at least 0.66 mm, at least 0.67 mm, at least 0.68 mm, at least 0.69 mm, at least 0.7 mm, at least 0.71 mm, at least 0.72 mm, at least 0.73 mm, at least 0.74 mm, at least 0.75 mm, at least 0.76 mm, at least 0.77 mm, at least 0.78 mm, at least 0.79 mm, at least 0.8 mm, at least 0.81 mm, at least 0.82 mm, at least 0.83 mm, at least 0.84 mm, at least 0.85 mm, at least 0.86 mm, at least 0.87 mm, at least 0.88 mm, at least 0.89 mm, at least 0.9 mm, at least 0.91 mm, at least 0.92 mm, at least 0.93 mm, at least 0.94 mm, at least 0.95 mm, at least 0.96 mm, at least 0.97 mm, at least 0.98 mm, at least 0.99 mm, at least 1.0 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, or at least 1.75 mm. In some embodiments, the ring height is the same for the plurality of rings. In some embodiments, at least one ring is a different height than another ring in the plurality of rings; the head can comprise any combination of different (e.g., at least 2, at least 3, at least 4, at least 5) ring heights.

In some embodiments, the plurality of rings have a height of at most 0.5 mm, at most 0.51 mm, at most 0.52 mm, at most 0.53 mm, at most 0.54 mm, at most 0.55 mm, at most 0.56 mm, at most 0.57 mm, at most 0.58 mm, at most 0.59 mm, at most 0.6 mm, at most 0.61 mm, at most 0.62 mm, at most 0.63 mm, at most 0.64 mm, at most 0.65 mm, at most 0.66 mm, at most 0.67 mm, at most 0.68 mm, at most 0.69 mm, at most 0.7 mm, at most 0.71 mm, at most 0.72 mm, at most 0.73 mm, at most 0.74 mm, at most 0.75 mm, at most 0.76 mm, at most 0.77 mm, at most 0.78 mm, at most 0.79 mm, at most 0.8 mm, at most 0.81 mm, at most 0.82 mm, at most 0.83 mm, at most 0.84 mm, at most 0.85 mm, at most 0.86 mm, at most 0.87 mm, at most 0.88 mm, at most 0.89 mm, at most 0.9 mm, at most 0.91 mm, at most 0.92 mm, at most 0.93 mm, at most 0.94 mm, at most 0.95 mm, at most 0.96 mm, at most 0.97 mm, at most 0.98 mm, at most 0.99 mm, at most 1.0 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, or at most 1.75 mm.

In some embodiments, the plurality of rings, or at least a portion of the plurality of rings, are tapered, i.e., have sequentially reduced diameters towards one end, both ends, or from the middle of the plurality of rings. In some embodiments, the plurality of rings taper from a maximum diameter at the distal end of the head to a minimum diameter at the proximal end of the head (i.e., closer to the neck or cap). In some embodiments, the plurality of rings taper from a minimum diameter at the distal end of the head to a maximum diameter at the proximal end of the head. In some embodiments, the maximum diameter of the plurality of rings occurs at a middle ring(s) of the head and the diameters taper to a minimum diameter at the proximal and/or distal(s) end of the head. In some embodiments, the minimum diameter of the plurality of rings occurs at a middle ring(s) of the head and the diameters taper to a maximum diameter at the proximal and/or distal end(s) of the head. In some embodiments, the rings alternate between a minimum diameter and a maximum diameter.

In some embodiments, the axial shaft, or at least a portion of the axial shaft, is tapered, i.e., has sequentially reduced diameters towards one end, both ends, or from the middle of the axial shaft. In some embodiments, the axial shaft tapers from a maximum diameter at the distal end of the axial shaft to a minimum diameter at the proximal end of the axial shaft (i.e., closer to the neck or cap). In some embodiments, the axial shaft tapers from a minimum diameter at the distal end of the axial shaft to a maximum diameter at the proximal end of the axial shaft. In some embodiments, the maximum diameter of the axial shaft occurs in the middle of the head and the diameters taper to a minimum diameter at the proximal and/or distal(s) end of the axial shaft. In some embodiments, the minimum diameter of the axial shaft occurs in the middle of the head and the diameters taper to a maximum diameter at the proximal and/or distal end(s) of the axial shaft. In some embodiments, the axial shaft alternates between a minimum diameter and a maximum diameter.

In some embodiments, the plurality of rings have rounded edges, i.e., have eased, curved, and/or non-angular edge. In some embodiments, the rounding of the rings is manufactured using an abrasion method (e.g., bead blasting, sandpaper) and/or a mold (e.g., an injection mold). In some embodiments, the rounding of the rings facilitates insertion and withdrawal into the sample or subject. In some embodiments, the distance between the rounded end edge of a first ring to the rounded beginning edge of the next proximate second ring is at least 0.75 mm. In some embodiments, the distance between the rounded end edge of a first ring to the rounded beginning edge of the next proximate second ring is at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm. In some embodiments, the spacing between the rounded edges of sequential pair of rings is the same for all pairs in the head. In some embodiments, the spacing between the rounded edges of each sequential pair of rings is different for at least one of the pairs in the head; the head can comprise any combination of different (e.g., at least 2, at least 3, at least 4, at least 5) rounded ring edge spacing distances.

In some embodiments, the distance between the rounded end edge of a first ring to the rounded beginning edge of the next proximate second ring is at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, or at most 3 mm.

In some embodiments, at least one ring of the plurality of rings is an incomplete ring (see e.g., FIG. 1), i.e., is missing a portion of the ring. In some embodiments, the at least one incomplete ring can be included for swabs that are injection molded or otherwise molded. In some embodiments, the at least one incomplete ring can be a site for ejection pins to eject the molded swab from the mold. In some embodiments, the at least one incomplete ring is recessed so as to not result in abrasive or sharp features that would otherwise be introduced into the swab during ejection from the mold; such abrasive or sharp features are disadvantageous as they can directly press against and irritate the nasal cavity. In some embodiments, the at least one incomplete allows the remainder of the sample collection head and swab to be very smooth and avoid damage to patients. In some embodiments, the cross-section of the incomplete ring is a semicircle, a truncated circle, or a circle with one or more flat sides. In some embodiments, the incomplete ring does not comprise at least 5%, at least 10%, at least 15%, at least 20%, at least 25%, at least 30%, at least 35%, at least 40%, at least 45%, or at least 50% of a complete ring. In some embodiments, the incomplete ring exposes at least a portion of the axial shaft. In some embodiments, the 1^{st}, 2^{nd}, 3^{rd}, 4^{th}, 5^{th}, 6^{th}, 7^{th}, 8^{th}, 9^{th}, and/or 10^{th}, etc. ring (e.g., counting from the head end of the swab) is an incomplete ring. In some embodiments, the third ring (e.g., counting from the head end of the swab) is an incomplete ring.

In some embodiments, each incomplete ring exposes the axial shaft of the head for a distance of about 1.5 mm. In some embodiments, each incomplete ring exposes the axial shaft of the head for a distance of at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm.

In some embodiments, each incomplete ring exposes the axial shaft of the head for a distance of at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, or at most 4.0 mm.

In some embodiments, the distal end of the head (i.e., farthest from the neck and/or cap) is tipped with a bulb, e.g., to facilitate insertion into the nasal cavity and/or to prevent droplet formation on the sample collection head, which can lead to contamination of other samples. In some embodiments, the bulb is a sphere or a partial sphere. In some embodiments, the bulb is a hemisphere. In some embodiments, the bulb is an ellipsoid (i.e., a deformed sphere, e.g., a flattened or lengthened sphere) or a partial ellipsoid. In some embodiments, the bulb has a thickness (i.e., the distance from the proximal end of the bulb (e.g., end with the maximum diameter in the case of a hemisphere) to the distal end of the bulb) of 1.5 mm. In some embodiments, the bulb has a thickness of 0.1 mm-3.0 mm. In some embodiments, the bulb has a thickness of at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm.

In some embodiments, the bulb has a thickness of at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, or at most 3 mm.

In some embodiments, the bulb has a maximum diameter (e.g., closest to the next proximate ring) of 1.0 mm-4.0 mm. In some embodiments, the bulb has a maximum diameter of 2.5 mm. In some embodiments, the bulb has a maximum diameter of 1.0 mm. In some embodiments, the bulb has a maximum diameter of at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm.

In some embodiments, the bulb has a maximum diameter of at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, or at least most 4.0 mm. In some embodiments, the maximum diameter of the bulb is the same as the diameter of the next proximate ring. In some embodiments, the maximum diameter of the bulb is greater than the diameter of the next proximate ring. In some embodiments, the maximum diameter of the bulb is less than the diameter of the next proximate ring.

In some embodiments, a portion of the axial shaft connects the bulb to the next proximate ring. In embodiments comprising a hemispherical bulb, the bulb has a rounded edge (i.e., the edge with the maximum diameter or closest to the next proximate ring). In some embodiments, the distance between the rounded end edge of the bulb to the rounded beginning edge of the next proximate second ring is at least 0.75 mm. In some embodiments, the distance between the rounded end edge of the bulb to the rounded beginning edge of the next proximate second ring is 0.86 mm. In some embodiments, the distance between the rounded end edge of the bulb to the rounded beginning edge of the next proximate second ring is at least 0.1 mm, at least 0.15 mm, at least 0.2 mm, at least 0.25 mm, at least 0.3 mm, at least 0.35 mm, at least 0.4 mm, at least 0.45 mm, at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, or at least 3 mm.

In some embodiments, the distance between the rounded end edge of the bulb to the rounded beginning edge of the next proximate second ring is at most 0.1 mm, at most 0.15 mm, at most 0.2 mm, at most 0.25 mm, at most 0.3 mm, at most 0.35 mm, at most 0.4 mm, at most 0.45 mm, at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, or at most 3 mm.

In some embodiments, the spacing between the rounded edges of the bulb and the next proximate ring is the same as the spacing between the rounded edges of the plurality of rings. In some embodiments, the spacing between the rounded edges of the bulb and the next proximate ring is different from the spacing between the rounded edges of the plurality of rings. In some embodiments, the spacing between the rounded edges of the bulb and the next proximate ring is less than the spacing between the rounded edges of the plurality of rings. In some embodiments, the spacing between the rounded edges of the bulb and the next proximate ring is greater than the spacing between the rounded edges of the plurality of rings.

In some embodiments, the sample collection head comprises a spiral axis groove, i.e., a depression of similar dimensions to the rings disclosed herein that spirals around the axial shaft of the head. In some embodiments, the sample collection head comprises a spiral axis flange, i.e., an elevation or protrusion of similar dimensions to the rings disclosed herein that spirals around the axial shaft of the head. In some embodiments, the spiral axis groove or spiral axis flange is spaced 0.1 mm-3 mm apart. In some embodiments, the spiral axis groove or spiral axis flange is spaced 0.75 mm apart. In some embodiments, the spiral axis groove or spiral axis flange has a thickness of 0.1 mm-3 mm. In some embodiments, the spiral axis groove or spiral axis flange has a thickness of 1.0 mm. In some embodiments, the spiral axis groove or spiral axis flange has a diameter of 1.0 mm-4.0 mm. In some embodiments, the spiral axis groove or spiral axis flange has a diameter of 2.5 mm. In some embodiments, the spiral axis groove or spiral axis flange are tapered. In some embodiments, the spiral axis groove or spiral axis flange has rounded edges. In some embodiments, the sample collection head comprises any combination of a plurality of spaced annular rings, a spiral axis groove, or spiral axis flange. In some embodiments, the sample collection head comprises a plurality of rings and a spiral axis groove. In some embodiments, the sample collection head comprises a plurality of rings and a spiral axis flange. In some embodiments, the sample collection head comprises a spiral axis groove and a spiral axis flange. In some embodiments, the sample collection head comprises a plurality of rings, a spiral axis groove, and a spiral axis flange. In some embodiments, the sample collection head comprises a plurality of spiral axis grooves or a plurality of spiral axis flanges, e.g., 2, 3, 4, 5, 6, 7, 8, 9, 10, 11, 12, 13, 14, 15, 16, 17, 18, 19, 20, 21, 22, 23, 24, 25, or more spiral axis grooves or spiral axis flanges. In some embodiments, the plurality of spiral axis grooves or the plurality of spiral axis flanges are continuous or discontinuous, with the same or different spacing, thickness, and/or diameter.

In some embodiments, the head comprises a fibrous coating, which can also be referred to herein as “flocked”. As used herein, the term “fibrous material” refers to a plurality of discrete fibers. The fibers can be plant-derived or animal-derived, synthetic, or some combination of these. In plant-derived fibrous materials, the fibers are at least predominantly of plant origin, non-limiting examples of which include cotton, wood, Papyrus, rice, Ficus, mulberry, Yucca, sisal, bowstring hemp, and New Zealand flax. Additional non-limiting examples of fibrous coatings that can be found in traditional swabs include cotton, cellulose, rayon, and polyester. In some embodiments, the head comprises an absorbent or soluble material. In some embodiments, the head is non-flocked, e.g., does not comprise a fibrous coating. In some embodiments, the head comprises a polymer material, for example, a hydrophobic polymer. In certain embodiments the head, e.g., the non-flocked head, is fabricated from a polymer, e.g., polypropylene.

In some embodiments, the head is stippled, roughened, or textured. As used herein, the term “stipple” means to mark or engrave a surface with number small dots or specks. As used herein, the term “roughen” means to cause to have an uneven, irregular, non-smooth surface, e.g., through abrasion. As used herein, the term “texture” means to cause to have a rough or raised or engraved surface. The texture can comprise a regular or repeated pattern (e.g., parallel grooves, perpendicular grooves, circles such as concentric circles, etc.) or an irregular non-patterned configuration, or any combination of regular and irregular textures. In some embodiments, the texture can comprise nanotexture, e.g., with dimensions (e.g., depth, thickness, and/or length) ranging from 1 nm-100 μm (e.g., at least 1 nm, at least 10 nm, at least 100 nm, at least 1 μm, at least 10 μm, at least 20 μm, at least 30 μm, at least 40 μm, at least 50 μm, at least 60 μm, at least 70 μm, at least 80 μm, at least 90 μm, or at least 100 μm). In some embodiments, the stippling, roughening, or texturing is applied using bead-blasting. In some embodiments, the stippling, roughening, or texturing of the head increases the surface area of the head by at least 1%, at least 5%, at least 10%, at least 20%, at least 30%, at least 40%, at least 50%, at least 60%, at least 70%, at least 80, at least 90%, at least 100%, at least 150%, at least 200%, at least 250%, at least 300%, at least 350%, at least 400%, at least 450%, or at least 500%.

In some embodiments, the length of the sample collection head (e.g., the “proximal” end of the head, e.g., the first ring of the plurality of rings, to the from “distal” end of the head, e.g., the termination of the head at the bulb) is at least 15 mm. In some embodiments, the length of the head is about 19 mm. In some embodiments, the length of the head is about 1.6 cm (16 mm). In some embodiments, the length of the head is at least 5 mm, at least 5.5 mm, at least 6 mm, at least 6.5 mm, at least 7 mm, at least 7.5 mm, at least 8 mm, at least 8.5 mm, at least 9 mm, at least 9.5 mm, at least 10 mm, at least 10.5 mm, at least 11 mm, at least 11.5 mm, at least 12 mm, at least 12.5 mm, at least 13 mm, at least 13.5 mm, at least 14 mm, at least 14.5 mm, at least 15 mm, at least 15.5 mm, at least 16 mm, at least 16.5 mm, at least 17 mm, at least 17.5 mm, at least 18 mm, at least 18.5 mm, at least 19 mm, at least 19.5 mm, at least 20 mm, at least 20.5 mm, at least 21 mm, at least 21.5 mm, at least 22 mm, at least 22.5 mm, at least 23 mm, at least 23.5 mm, at least 24 mm, at least 24.5 mm, at least 25 mm, at least 25.5 mm, at least 26 mm, at least 26.5 mm, at least 27 mm, at least 27.5 mm, at least 28 mm, at least 28.5 mm, at least 29 mm, at least 29.5 mm, at least 30 mm, at least 30.5 mm, at least 31 mm, at least 31.5 mm, at least 32 mm, at least 32.5 mm, at least 33 mm, at least 33.5 mm, at least 34 mm, at least 34.5 mm, at least 35 mm, at least 35.5 mm, at least 36 mm, at least 36.5 mm, at least 37 mm, at least 37.5 mm, at least 38 mm, at least 38.5 mm, at least 39 mm, at least 39.5 mm, at least 40 mm, at least 40.5 mm, at least 41 mm, at least 41.5 mm, at least 42 mm, at least 42.5 mm, at least 43 mm, at least 43.5 mm, at least 44 mm, at least 44.5 mm, at least 45 mm, at least 45.5 mm, at least 46 mm, at least 46.5 mm, at least 47 mm, at least 47.5 mm, at least 48 mm, at least 48.5 mm, at least 49 mm, at least 49.5 mm, or at least 50 mm.

In some embodiments, the length of the head is at most 5 mm, at most 5.5 mm, at most 6 mm, at most 6.5 mm, at most 7 mm, at most 7.5 mm, at most 8 mm, at most 8.5 mm, at most 9 mm, at most 9.5 mm, at most 10 mm, at most 10.5 mm, at most 11 mm, at most 11.5 mm, at most 12 mm, at most 12.5 mm, at most 13 mm, at most 13.5 mm, at most 14 mm, at most 14.5 mm, at most 15 mm, at most 15.5 mm, at most 16 mm, at most 16.5 mm, at most 17 mm, at most 17.5 mm, at most 18 mm, at most 18.5 mm, at most 19 mm, at most 19.5 mm, at most 20 mm, at most 20.5 mm, at most 21 mm, at most 21.5 mm, at most 22 mm, at most 22.5 mm, at most 23 mm, at most 23.5 mm, at most 24 mm, at most 24.5 mm, at most 25 mm, at most 25.5 mm, at most 26 mm, at most 26.5 mm, at most 27 mm, at most 27.5 mm, at most 28 mm, at most 28.5 mm, at most 29 mm, at most 29.5 mm, at most 30 mm, at most 30.5 mm, at most 31 mm, at most 31.5 mm, at most 32 mm, at most 32.5 mm, at most 33 mm, at most 33.5 mm, at most 34 mm, at most 34.5 mm, at most 35 mm, at most 35.5 mm, at most 36 mm, at most 36.5 mm, at most 37 mm, at most 37.5 mm, at most 38 mm, at most 38.5 mm, at most 39 mm, at most 39.5 mm, at most 40 mm, at most 40.5 mm, at most 41 mm, at most 41.5 mm, at most 42 mm, at most 42.5 mm, at most 43 mm, at most 43.5 mm, at most 44 mm, at most 44.5 mm, at most 45 mm, at most 45.5 mm, at most 46 mm, at most 46.5 mm, at most 47 mm, at most 47.5 mm, at most 48 mm, at most 48.5 mm, at most 49 mm, at most 49.5 mm, or at most 50 mm. In some embodiments of any of the aspects, the combined length of the head and the neck is at most 25 mm, at most 30 mm, at most 35 mm, at most 40 mm, at most 45 mm, at most 50 mm, at most 55 mm, at most 60 mm, at most 65 mm, at most 70 mm, at most 75 mm, at most 80 mm, at most 85 mm, at most 90 mm, at most 95 mm, at most 100 mm, at most 105 mm, at most 110 mm, at most 115 mm, at most 120 mm, at most 125 mm, at most 130 mm, at most 135 mm, at most 140 mm, at most 145 mm, or at most 150 mm.

### Neck

In some embodiments, the swab further comprises a neck. In some embodiments, the neck connects the sample collection head to the cap. In some embodiments, the neck comprises a rod (see e.g., FIG. 1). In some embodiments, the cross-section of the neck is a circle, a semicircle, a truncated circle, or a circle with one or more flat sides. In some embodiments, the cross-section of the neck is a circle. In some embodiments, the neck comprises a cylindrical rod. In some embodiments, the neck comprises a rod with a polygonal cross section, e.g., a cross-section in the shape of a triangle, a square, a quadrilateral, a trapezoid, a pentagon, a hexagon, or a polygon with at least 7, 8, 9, 10, 11, 12, 13, 14, 15, 16, 17, 18, 19, 20 or more sides. In some embodiments, at least one side of the cross-section of the neck comprises a convex and/or concave curve. In some embodiments, the cross section of the neck is a rotationally symmetric shape. In some embodiments, the cross section of the neck is an asymmetric shape. In some embodiments, the neck cross-section is the same for the entirety of the neck. In some embodiments, the neck cross-section is different for at least one portion of the neck; the neck can comprise any combination of different (e.g., at least 2, at least 3, at least 4, at least 5) neck cross-sections. In some embodiments, the neck tapers from a maximum diameter (e.g., towards the cap) to a smaller diameter (e.g., towards the head). In some embodiments, the maximum diameter of the neck is the same as the minimum diameter of the cap. In some embodiments, the maximum diameter of the neck is less than the minimum diameter of the cap. In some embodiments, the maximum diameter of the neck is greater than the minimum diameter of the cap. In some embodiments, the minimum diameter of the neck is the same as the maximum diameter of the axial shaft of the sample collection head. In some embodiments, the minimum diameter of the neck is less than as the maximum diameter of the axial shaft of the sample collection head. In some embodiments, the minimum diameter of the neck is greater than the maximum diameter of the axial shaft of the sample collection head.

In some embodiments, the rate of the tapering of the neck is constant and/or continuous. In some embodiments, the rate of the tapering of the neck is non-constant and/or discontinuous. In some embodiments, the neck comprises a plurality of sections (e.g., 2, 3, 4, 5, 6, 7, 8, 9, 10 or more) each with a different rate of tapering and/or no tapering. In some embodiments, each section of the neck is continuous with the next proximate section, i.e., a first section (farther from the head) of the neck has a minimum diameter that is the same as the maximum diameter of the next proximate second section (closer to the head) of the neck.

In some embodiments, the neck (or any section of the neck) has a maximum diameter (e.g., towards the cap) of about 1.0 mm-4.0 mm. In some embodiments, the neck (or any section of the neck) has a maximum diameter of about 1.5 mm. In some embodiments, the neck (or any section of the neck) has a maximum diameter of at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, at least 3.5 mm, at least 3.55 mm, at least 3.6 mm, at least 3.65 mm, at least 3.7 mm, at least 3.75 mm, at least 3.8 mm, at least 3.85 mm, at least 3.9 mm, at least 3.95 mm, or at least 4.0 mm.

In some embodiments, the neck (or any section of the neck) has a maximum diameter of at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, at most 3.5 mm, at most 3.55 mm, at most 3.6 mm, at most 3.65 mm, at most 3.7 mm, at most 3.75 mm, at most 3.8 mm, at most 3.85 mm, at most 3.9 mm, at most 3.95 mm, or at most 4.0 mm.

In some embodiments, the neck (or any section of the neck) has a minimum diameter (e.g., towards the head) of about 0.5 mm-3.5 mm. In some embodiments, the neck (or any section of the neck) has a minimum diameter of 1.2 mm. In some embodiments, the neck (or any section of the neck) has a minimum diameter of at least 0.5 mm, at least 0.55 mm, at least 0.6 mm, at least 0.65 mm, at least 0.7 mm, at least 0.75 mm, at least 0.8 mm, at least 0.85 mm, at least 0.9 mm, at least 0.95 mm, at least 1 mm, at least 1.05 mm, at least 1.1 mm, at least 1.15 mm, at least 1.2 mm, at least 1.25 mm, at least 1.3 mm, at least 1.35 mm, at least 1.4 mm, at least 1.45 mm, at least 1.5 mm, at least 1.55 mm, at least 1.6 mm, at least 1.65 mm, at least 1.7 mm, at least 1.75 mm, at least 1.8 mm, at least 1.85 mm, at least 1.9 mm, at least 1.95 mm, at least 2 mm, at least 2.05 mm, at least 2.1 mm, at least 2.15 mm, at least 2.2 mm, at least 2.25 mm, at least 2.3 mm, at least 2.35 mm, at least 2.4 mm, at least 2.45 mm, at least 2.5 mm, at least 2.55 mm, at least 2.6 mm, at least 2.65 mm, at least 2.7 mm, at least 2.75 mm, at least 2.8 mm, at least 2.85 mm, at least 2.9 mm, at least 2.95 mm, at least 3 mm, at least 3.05 mm, at least 3.1 mm, at least 3.15 mm, at least 3.2 mm, at least 3.25 mm, at least 3.3 mm, at least 3.35 mm, at least 3.4 mm, at least 3.45 mm, or at least 3.5 mm.

In some embodiments, the neck (or any section of the neck) has a minimum diameter of at most 0.5 mm, at most 0.55 mm, at most 0.6 mm, at most 0.65 mm, at most 0.7 mm, at most 0.75 mm, at most 0.8 mm, at most 0.85 mm, at most 0.9 mm, at most 0.95 mm, at most 1 mm, at most 1.05 mm, at most 1.1 mm, at most 1.15 mm, at most 1.2 mm, at most 1.25 mm, at most 1.3 mm, at most 1.35 mm, at most 1.4 mm, at most 1.45 mm, at most 1.5 mm, at most 1.55 mm, at most 1.6 mm, at most 1.65 mm, at most 1.7 mm, at most 1.75 mm, at most 1.8 mm, at most 1.85 mm, at most 1.9 mm, at most 1.95 mm, at most 2 mm, at most 2.05 mm, at most 2.1 mm, at most 2.15 mm, at most 2.2 mm, at most 2.25 mm, at most 2.3 mm, at most 2.35 mm, at most 2.4 mm, at most 2.45 mm, at most 2.5 mm, at most 2.55 mm, at most 2.6 mm, at most 2.65 mm, at most 2.7 mm, at most 2.75 mm, at most 2.8 mm, at most 2.85 mm, at most 2.9 mm, at most 2.95 mm, at most 3 mm, at most 3.05 mm, at most 3.1 mm, at most 3.15 mm, at most 3.2 mm, at most 3.25 mm, at most 3.3 mm, at most 3.35 mm, at most 3.4 mm, at most 3.45 mm, or at most 3.5 mm.

In some embodiments, the length of the neck is about 20 mm-100 mm. In some embodiments, the length of the neck is at least 50 mm. In some embodiments, the length of the neck is at least 25 mm. In some embodiments of any of the aspects, the length of the neck is at least 20 mm, at least 25 mm, at least 30 mm, at least 35 mm, at least 40 mm, at least 45 mm, at least 50 mm, at least 55 mm, at least 60 mm, at least 65 mm, at least 70 mm, at least 75 mm, at least 80 mm, at least 85 mm, at least 90 mm, at least 95 mm, or at least 100 mm. In some embodiments of any of the aspects, the length of the neck is at most 20 mm, at most 25 mm, at most 30 mm, at most 35 mm, at most 40 mm, at most 45 mm, at most 50 mm, at most 55 mm, at most 60 mm, at most 65 mm, at most 70 mm, at most 75 mm, at most 80 mm, at most 85 mm, at most 90 mm, at most 95 mm, or at most 100 mm.

In some embodiments, the combined length of the head and the neck is about 25 mm-150 mm. In some embodiments, the combined length of the head and the neck is at least 75 mm. In some embodiments, the combined length of the head and the neck is at least 45 mm. In some embodiments of any of the aspects, the combined length of the head and the neck is at least 25 mm, at least 30 mm, at least 35 mm, at least 40 mm, at least 45 mm, at least 50 mm, at least 55 mm, at least 60 mm, at least 65 mm, at least 70 mm, at least 75 mm, at least 80 mm, at least 85 mm, at least 90 mm, at least 95 mm, at least 100 mm, at least 105 mm, at least 110 mm, at least 115 mm, at least 120 mm, at least 125 mm, at least 130 mm, at least 135 mm, at least 140 mm, at least 145 mm, or at least 150 mm. In some embodiments of any of the aspects, the combined length of the head and the neck is at most 25 mm, at most 30 mm, at most 35 mm, at most 40 mm, at most 45 mm, at most 50 mm, at most 55 mm, at most 60 mm, at most 65 mm, at most 70 mm, at most 75 mm, at most 80 mm, at most 85 mm, at most 90 mm, at most 95 mm, at most 100 mm, at most 105 mm, at most 110 mm, at most 115 mm, at most 120 mm, at most 125 mm, at most 130 mm, at most 135 mm, at most 140 mm, at most 145 mm, or at most 150 mm.

### Threaded Portion

In some embodiments, the swab further comprises a threaded portion. As used herein, e.g., in reference to the swab and/or collection tube, the term “threaded portion” refers to a cylindrical portion comprising raised helical thread(s). In some embodiments, the threaded portion of the swab comprises 1, 2, 3, 4, 5, or more threads, which can be continuous or discontinuous. In some embodiments, the thread(s) wraps clockwise or counterclockwise around the swab, e.g., when viewed from the head end of the swab. In some embodiments, the threaded portion of the swab comprises a geometry that interfaces with the geometry of the threaded portion of the container tube. In some embodiments, the swab comprises an externally threaded portion that interfaces with an internally threaded portion of the container tube. In some embodiments, the swab comprises an internally threaded portion that interfaces with an externally threaded portion of the container tube. In some embodiments, the threaded portion is located between the neck and the cap (see FIG. 1). In some embodiments, the threaded portion is an integral component of the cap. In some embodiments, the threaded portion of the swab comprises external ridges (also known as flanges). In some embodiments, the threaded portion of the swab comprises external grooves. In some embodiments, the threaded portion of the swab comprises internal ridges (also known as flanges). In some embodiments, the threaded portion of the swab comprises internal grooves. In some embodiments, the threaded portion of the swab comprises (or is replaced by) an external or internal geometric feature to permit snapping, holding in place (e.g., a bayonet mount, interference fit, etc.), and/or sealing the swab and biological sample within the container tube, and the container tube comprises the corresponding geometry to interface with the swab.

In some embodiments, the geometry of the threaded portion of the swab matches the geometry of the threaded portion of the container tube. In these embodiments, one or more of the pitch, the direction, the number of threads, the dimensions of the threads, etc. can match between the swab and the container tube. In some implementations, the threaded portion of the swab is made to lock onto the container tube. In these embodiments, additional force or force applied at an angle to the axis of the swab and the tube is required in order to unscrew the swab from the container tube. In some embodiments, the threaded portion of the swab is designed so that the swab can be removed (e.g., unscrewed) from the container tube with minimal actuation force. In some embodiments, the threaded portion of the swab and/or the threaded portion of the container tube contain an O-ring or gasket to aid in forming a fluid-tight or substantially fluid-tight seal between the threaded portion of the swab and the threaded portion of the container tube. The O-ring or gasket thus aids in stopping liquid from leaking from the container tube.

### Cap

In some embodiments, the swab further comprises a cap, e.g., at the proximal head of the swab. In some embodiments, the distal edge of the cap (i.e., closer to the head of the swab) seals with the opening of a container tube, e.g., when the threaded portion of the swab is screwed into or onto the threaded portion of the container tube. In some embodiments, the diameter of the distal edge of the cap is greater than the diameter of the opening of the container tube. In some embodiments, the diameter of the distal edge of the cap is the same as the diameter of the opening of the container tube. In some embodiments, the cap is integrally and/or monolithically formed with the rest of the swab. In other embodiments, the cap is a physically separate component that can be removably attached to the swab, for example to the threaded portion of the swab or the neck of the swab. In other embodiments, the cap is a physically separate component that can be permanently attached to the swab, for example, to the threaded portion of the swab or the neck of the swab. The cap can be attached to the swab using a variety of techniques, including an adhesive, a weld, a heat stake, or other chemical or physical bonding techniques. In various embodiments, each of the cap, threaded portion, neck, and/or head can be formed of a single molded part (e.g., a unitary part or item) or as separate parts, in any combination or permutation. For example, the neck and head can be formed as a single molded part (e.g., a unitary part or item) and be attached to a separate cap.

In some embodiments, the cap comprises a structure and/or configuration adapted to interface with an automation device (e.g., a tube capper or decapper machine). In general, the cap can have any structure that corresponds with any known or future developed automation device. For example, in some embodiments, the cap comprises a hollow internal portion, e.g., that interfaces with an automated device. In some embodiments, an outer surface of the cap (e.g., top surface, circumferential surface, side surface) interfaces with an automation device. In some embodiments, the proximal end of the cap (i.e., farther away from the head of the swab) defines an opening leading to hollow internal portion of the cap. In these embodiments, the hollow internal portion of the cap can thus be open to the exterior of the cap. In some embodiments, the cap comprises a hollow cylinder. In some embodiments, the cap is defined by an outer cross-section (i.e., the external shape of the cap) and an inner cross-section (i.e., the internal shape of the hollow portion). In some embodiments, the outer and/or inner cross-section of the cap is a circle, a semicircle, a truncated circle, or a circle with one or more flat sides. In some embodiments, the outer and/or inner cross-section of the cap is a circle. In some embodiments, the outer and/or inner cross-section of the cap comprises a polygonal cross section, e.g., a cross-section in the shape of a triangle, a square, a quadrilateral, a trapezoid, a pentagon, a hexagon, a star, or a polygon with at least 7, 8, 9, 10, 11, 12, 13, 14, 15, 16, 17, 18, 19, 20 or more sides. In some embodiments, at least one side of the outer and/or inner cross-section of the cap comprises a convex and/or concave curve. In some embodiments, the outer and/or inner cross section of the cap is a rotationally symmetric shape. In some embodiments, the outer and/or inner cross section of the cap is an asymmetric shape. In some embodiments, the outer and/or inner cap cross-section is the same for the entirety of the cap. In some embodiments, the outer and/or inner cap cross-section is different for at least one portion of the cap; the cap can comprise any combination of different (e.g., at least 2, at least 3, at least 4, at least 5) cap cross-sections. In some embodiments, the outer and inner cap cross-sections of the cap are the same. In some embodiments, the outer and inner cap cross-sections of the cap are different.

In some embodiments, the cap comprises at least one (e.g., 1, 2, 3, 4, 5, or more) internal groove(s). In some embodiments, the cap comprises at least one (e.g., 1, 2, 3, 4, 5, or more) internal ridge(s). In some embodiments, the cap comprises at least one (e.g., 1, 2, 3, 4, 5, or more) external groove(s). In some embodiments, the cap comprises at least one (e.g., 1, 2, 3, 4, 5, or more) external ridge(s). In some embodiments, the internal or external groove(s) or internal ridge(s) are parallel with the axial shaft of the swab.

In some embodiments, the cap can interface with an automated device. In some embodiments, the automated device can move, control, manipulate, etc. the swab after interfacing with the cap. In some embodiments, a portion of an automated device can extend into the hollow internal portion of the cap. In some embodiments, hollow portion and the internal groove(s) or internal ridge(s) permit the cap to interface with an automated device. In some embodiments, the automated device is a tube capper and decapper machine. In some embodiments, the cap can be adjusted to fit any standard or custom tube that is compatible with the SBS 24-well format, the SBS 48-well format, the SBS 96-well format, or any combination thereof. In some embodiments, the cap can be adjusted for any automation format.

In some embodiments, the cap can be used as a handle by the person using the swab. For example, a user can grasp the swab by the handle to control the swab and insert the head into the user's anterior nares. In some embodiments, the swab includes a handle portion that can extend from the cap of the swab. The handle portion can be removably coupled to the cap. In these embodiments, the user can remove the handle portion from the cap after the sample has been obtained. In some of these embodiments, the handle portion can be removed by the user via manual force. For example, the user could snap off, twist off, pull off, or otherwise remove the handle portion from the cap. The handle portion can also include a guard that can aid in preventing the user's fingers from slipping off of the handle portion.

The handle portion can include a breakpoint, which is a location along the handle portion with a minimal diameter, such that application of force separates the handle portion from the cap at the breakpoint. In some embodiments, a break at the breakpoint can be accomplished by a single direction bend. In some embodiments, a break at the breakpoint can be accomplished by torsion (i.e., twisting). In some embodiments, a break at the breakpoint can be accomplished by a single direction bend combined with torsion, before and/or after the bend or at the same time as the bend. The handle portion can include multiple breakpoints. In some embodiments, the cross-section of the breakpoint is a circle, a semicircle, a truncated circle, or a circle with one or more flat sides. In some embodiments, the cross-section of the breakpoint is a circle. In some embodiments, the breakpoint has a polygonal cross section, e.g., a cross-section in the shape of a triangle, a square, a quadrilateral, a trapezoid, a pentagon, a hexagon, or a polygon with at least 7, 8, 9, 10, 11, 12, 13, 14, 15, 16, 17, 18, 19, 20 or more sides. In some embodiments, at least one side of the cross-section of the breakpoint comprises a convex and/or concave curve. In some embodiments, the cross section of the breakpoint is a rotationally symmetric shape. In some embodiments, the cross section of the breakpoint is an asymmetric shape. In some embodiments, the breakpoint cross-section is the same for the entirety of the breakpoint. In some embodiments, the breakpoint cross-section is different for at least one portion of the breakpoint; the breakpoint can comprise any combination of different (e.g., at least 2, at least 3, at least 4, at least 5) breakpoint cross-sections.

The handle portion can also be configured to be removed automatically when the cap is screwed onto the container tube. For example, the breakpoint can be positioned so that when threaded portion of the swab engages with the threaded portion of the container tube, the container tube imparts a force on the handle portion, resulting in a break at the breakpoint. In these embodiments, handle portion may extend from the threaded portion of the swab instead of the cap. The handle portion can also be configured to be removed automatically when the cap is screwed onto the container tube without the presence of a breakpoint. In any of these embodiments, the handle portion can be configured to be break away from the swab once the cap has been screwed onto the container tube with an appropriate amount of force. In these embodiments, the breaking away of the handle portion indicates that no more screwing of the cap onto the container tube is needed, thereby aiding in preventing overtightening or under-tightening of the cap onto the container tube.

### Materials

In some embodiments, the swab material exhibits at least one of the following characteristics: (1) It is sufficiently rigid for collection of cells (e.g., from the back of the throat). (2) It is sufficiently flexible for safety of use. (3) It collects adequate sample from the patient for subsequent tests (e.g., for viral infection). (4) It withstands the rigors of sterilization/disinfection without a) structural weakening, or b) chemically interfering with PCR testing. (5) It is compatible with standard PCR testing and nucleic acid extraction technologies. In some embodiments, the swab material is biodegradable and/or water-soluble.

In some embodiments, the swab is constructed from a semi-flexible material, such as polypropylene, polycarbonate, thermoplastic elastomers (TPE), rubber, polyester fiber, acrylonitrile butadiene styrene (ABS), acrylic, polyetherimide, ionomer, acetal copolymer, polyurethane, polystyrene, nylon, and the like, or any combination thereof. In some embodiments, the swab material is a flexible polymer. In some embodiments, the swab material is a solid material (i.e., non-porous). In some embodiments, the swab material is a foam. In some embodiments, the swab material is hydrophobic. In some embodiments, the swab material is a porous material. In some embodiments, all of the components of the swab (e.g., head, neck, threaded portion, and/or cap) comprise the same material. In some embodiments, at least one component of the swab (e.g., head, neck, threaded portion, and/or cap) is made from a different material from the remainder of the swab. In some embodiments, the swab comprises at least 2 (e.g., 2, 3, 4, 5, or more) materials as described herein. As a non-limiting example, a swab comprising at least two materials can be accomplished using injection molding (i.e., overmolding). Overmolding is a process wherein a single part is created using two or more different materials in combination. Typically, the first material, sometimes referred to as the substrate, is partially or fully covered by subsequent materials (i.e., overmold materials) during the manufacturing process.

In some embodiments, swabs (or portions thereof, e.g., cap, threaded portion, neck, and/or head) can be formed from any of the materials described herein and can also have a length of any of the following: at least 20 mm, at least 25 mm, at least 30 mm, at least 35 mm, at least 40 mm, at least 45 mm, at least 50 mm, at least 55 mm, at least 60 mm, at least 65 mm, at least 70 mm, at least 75 mm, at least 80 mm, at least 85 mm, at least 90 mm, at least 95 mm, at least 100 mm, at most 20 mm, at most 25 mm, at most 30 mm, at most 35 mm, at most 40 mm, at most 42 mm, at most 45 mm, at most 49 mm, at most 50 mm, at most 55 mm, at most 60 mm, at most 65 mm, at most 70 mm, at most 73 mm, at most 75 mm, at most 80 mm, at most 82 mm, at most 85 mm, at most 90 mm, at most 95 mm, at most 100 mm, at most 105 mm, at most 110 mm, at most 115 mm, at most 120 mm, at most 125 mm, at most 130 mm, in a range from 1 mm to 100 mm, in a range from 5 mm to 95 mm, in a range from 10 mm to 90 mm, in a range from 15 mm to 85 mm, in a range from 20 mm to 80 mm, in a range from 25 mm to 75 mm, in a range from 30 mm to 70 mm, in a range from 35 mm to 65 mm, in a range from 40 mm to 60 mm, and in a range from 45 mm to 55 mm.

In some embodiments, the swab material comprises polypropylene. In some embodiments, the polypropylene swab material comprises Flint Hills Resources™ (FHR) P5M4R polypropylene copolymer. In some embodiments, the polypropylene swab material is medical grade. In some embodiments, the polypropylene swab material comprises a random copolymer for injection molding. In some embodiments, the swab material exhibits the following features: autoclave sterilizable; E-beam sterilizable; ethylene oxide sterilizable; no animal derived components; and radiation sterilizable.

In some embodiments, the swab material does not comprise nylon. In some embodiments, the swab material does not comprise polystyrene. In some embodiments, the swab material is hydrophobic. In some embodiments, at least one component of the swab is a different material than other components of the swab. In some embodiments, the swab comprises 1, 2, 3, 4, 5, or more different materials. As a non-limiting example, the sample collection head comprises a first material, and the cap comprises a second, different material.

In some embodiments, the swab material has a flexural modulus of about 500 megapascals (MPa) to 800 MPa. As used herein, the term “flexural modulus” (also referred to as bending modulus) is the ratio of stress to strain in flexural deformation, or the tendency for a material to resist bending. In some embodiments, the swab material has a tangent flexural modulus of about 790 MPa. In some embodiments, the swab material has a flexural modulus of about 500 MPa to 2000 MPa. In some embodiments, the swab material has a flexural modulus of about 100 MPa to 5000 MPa.

In some embodiments, the swab material has a flexural modulus of at least 100 MPa, at least 150 MPa, at least 200 MPa, at least 250 MPa, at least 300 MPa, at least 350 MPa, at least 400 MPa, at least 450 MPa, at least 500 MPa, at least 500 MPa, at least 510 MPa, at least 520 MPa, at least 530 MPa, at least 540 MPa, at least 550 MPa, at least 560 MPa, at least 570 MPa, at least 580 MPa, at least 590 MPa, at least 600 MPa, at least 610 MPa, at least 620 MPa, at least 630 MPa, at least 640 MPa, at least 650 MPa, at least 660 MPa, at least 670 MPa, at least 680 MPa, at least 690 MPa, at least 700 MPa, at least 710 MPa, at least 720 MPa, at least 730 MPa, at least 740 MPa, at least 750 MPa, at least 760 MPa, at least 770 MPa, at least 780 MPa, at least 790 MPa, at least 800 MPa, at least 850 MPa, at least 900 MPa, at least 950 MPa, at least 1000 MPa, at least 1050 MPa, at least 1100 MPa, at least 1150 MPa, at least 1200 MPa, at least 1250 MPa, at least 1300 MPa, at least 1350 MPa, at least 1400 MPa, at least 1450 MPa, at least 1500 MPa, at least 1550 MPa, at least 1600 MPa, at least 1650 MPa, at least 1700 MPa, at least 1750 MPa, at least 1800 MPa, at least 1850 MPa, at least 1900 MPa, at least 1950 MPa, at least 2000 MPa, at least 2000 MPa, at least 2100 MPa, at least 2200 MPa, at least 2300 MPa, at least 2400 MPa, at least 2500 MPa, at least 2600 MPa, at least 2700 MPa, at least 2800 MPa, at least 2900 MPa, at least 3000 MPa, at least 3100 MPa, at least 3200 MPa, at least 3300 MPa, at least 3400 MPa, at least 3500 MPa, at least 3600 MPa, at least 3700 MPa, at least 3800 MPa, at least 3900 MPa, at least 4000 MPa, at least 4100 MPa, at least 4200 MPa, at least 4300 MPa, at least 4400 MPa, at least 4500 MPa, at least 4600 MPa, at least 4700 MPa, at least 4800 MPa, at least 4900 MPa, or at least 5000 MPa.

In some embodiments, the swab material has a flexural modulus of at most 100 MPa, at most 150 MPa, at most 200 MPa, at most 250 MPa, at most 300 MPa, at most 350 MPa, at most 400 MPa, at most 450 MPa, at most 500 MPa, at most 500 MPa, at most 510 MPa, at most 520 MPa, at most 530 MPa, at most 540 MPa, at most 550 MPa, at most 560 MPa, at most 570 MPa, at most 580 MPa, at most 590 MPa, at most 600 MPa, at most 610 MPa, at most 620 MPa, at most 630 MPa, at most 640 MPa, at most 650 MPa, at most 660 MPa, at most 670 MPa, at most 680 MPa, at most 690 MPa, at most 700 MPa, at most 710 MPa, at most 720 MPa, at most 730 MPa, at most 740 MPa, at most 750 MPa, at most 760 MPa, at most 770 MPa, at most 780 MPa, at most 790 MPa, at most 800 MPa, at most 850 MPa, at most 900 MPa, at most 950 MPa, at most 1000 MPa, at most 1050 MPa, at most 1100 MPa, at most 1150 MPa, at most 1200 MPa, at most 1250 MPa, at most 1300 MPa, at most 1350 MPa, at most 1400 MPa, at most 1450 MPa, at most 1500 MPa, at most 1550 MPa, at most 1600 MPa, at most 1650 MPa, at most 1700 MPa, at most 1750 MPa, at most 1800 MPa, at most 1850 MPa, at most 1900 MPa, at most 1950 MPa, at most 2000 MPa, at most 2000 MPa, at most 2100 MPa, at most 2200 MPa, at most 2300 MPa, at most 2400 MPa, at most 2500 MPa, at most 2600 MPa, at most 2700 MPa, at most 2800 MPa, at most 2900 MPa, at most 3000 MPa, at most 3100 MPa, at most 3200 MPa, at most 3300 MPa, at most 3400 MPa, at most 3500 MPa, at most 3600 MPa, at most 3700 MPa, at most 3800 MPa, at most 3900 MPa, at most 4000 MPa, at most 4100 MPa, at most 4200 MPa, at most 4300 MPa, at most 4400 MPa, at most 4500 MPa, at most 4600 MPa, at most 4700 MPa, at most 4800 MPa, at most 4900 MPa, or at most 5000 MPa.

In another aspect, described herein is a swab constructed from a water-soluble or biodegradable material. In some embodiments, the swab material is biodegradable and water-soluble. In some embodiments, the swab material is biodegradable. In some embodiments, the swab material is water-soluble. In some embodiments, the swab material is a foam. In some embodiments, the swab material is a porous material. Non-limiting examples of biodegradable swab materials include a bio-based plastic, polyhydroxyalkanoate (PHA), polylactic acid (PLA), starch blend, cellulose-based plastic, lignin-based polymer composite, a petroleum-based plastic, polyglycolic acid (PGA), polybutylene succinate (PBS), polycaprolactone (PCL), poly(vinyl alcohol) (PVA, PVOH), or polybutylene adipate terephthalate (PBAT). In some embodiments the swab material comprises polyvinyl alcohol or a derivative polymer such as polyvinyl acetals, polyvinyl butyral (PVB), or polyvinyl formal (PVF). In some embodiments, the swab material comprises Kuraray MOWIFLEX™ C17 or C30 materials, which are PVA variants. In some embodiments, the material consists essentially of polyvinyl alcohol. In some embodiments, the material (e.g., polyvinyl alcohol) does not interfere with downstream applications (e.g., PCR, qPCR, RT-qPCR, isothermal amplification, RPA, etc.). In some embodiments, the sample collection head comprises a first material, and the remainder of the swab (e.g. neck, threaded portion, and/or cap) comprises a second material. As a non-limiting example, the sample collection head comprises a water-soluble and/or biodegradable material and the remainder of the swab comprises a flexible polymer. As a non-limiting example, the sample collection head comprises PVA and the remainder of the swab comprises polypropylene.

### Kits

Another aspect of the technology described herein relates to kits for collecting samples using the swabs as described herein. Described herein are kit components that can be included in one or more of the kits described herein.

In some embodiments, the kit comprises a swab as described herein. In some embodiments, the kit comprises a swab comprising a sample collection head, a neck, a threaded portion, and a cap. In some embodiments, the kit comprises 1, 2, 3, 4, 5, 6, 7, 8, 9, 10, 11, 12, 13, 14, 15, 16, 17, 18, 19, 20 or more swabs as described herein.

In some embodiments, the kit further comprises a container tube as described herein. In some embodiments, the kit comprises 1, 2, 3, 4, 5, 6, 7, 8, 9, 10, 11, 12, 13, 14, 15, 16, 17, 18, 19, 20 or more container tubes as described herein.

In some embodiments, the kit further comprises an effective amount of sample transport media. As will be appreciated by one of skill in the art, the sample transport media can be supplied in a lyophilized or dried form or a concentrated liquid form that can diluted or suspended in liquid prior to use with the swab. Preferred formulations include those that are non-toxic to the samples (e.g., cells, bacteria, viruses) and/or does not affect growth rate or viability. When the sample transport media is provided in a liquid solution, the liquid solution preferably is an aqueous solution, with a sterile aqueous solution being preferred. The sample transport media can be supplied in aliquots or in unit doses. In some embodiments of any of the aspects, transport media preserves the sample components (e.g., cellular, bacterial, or viral nucleic acids or polypeptides) nucleic acid between the time of sample collection and downstream applications.

In some embodiments of any of the aspects, the sample transport media comprises a viral transport media (VTM). The constituents of suitable viral transport media are designed to provide an isotonic solution containing protective protein, antibiotics to control microbial contamination, and one or more buffers to control the pH. Isotonicity, however, is not an absolute requirement; some highly successful transport media contain hypertonic solutions of sucrose. Liquid transport media are used primarily for transporting swabs or materials released into the medium from a collection swab. Liquid media may be added to other specimens when inactivation of the viral agent is likely and when the resultant dilution is acceptable. A suitable VTM for use in collecting throat and nasal swabs from human patients is prepared as follows: (1) add 10 g veal infusion broth and 2 g bovine albumin fraction V to sterile distilled water (to 400 ml); (2) add 0.8 ml gentamicin sulfate solution (50 mg/ml) and 3.2 ml amphotericin B (250 μg/ml); and (3) sterilize by filtration. Additional non-limiting examples of viral transport media include COPAN Universal Transport Medium; Eagle Minimum Essential Medium (E-MEM); Transport medium 199; and PBS-Glycerol transport medium. see e.g., Johnson, Transport of Viral Specimens, CLINICAL MICROBIOLOGY REVIEWS, April 1990, p. 120-131; Collecting, preserving and shipping specimens for the diagnosis of avian influenza A(H5N1) virus infection, Guide for field operations, October 2006.

In some embodiments, the components described herein can be provided singularly or in any combination as a kit. Such a kit includes the components described herein, e.g., a swab, a container tube, and/or sample transport media, as described throughout the specification, or any combination thereof. Such kits can optionally include one or more agents that permit the detection of cellular, bacterial, or viral nucleic acids or polypeptides in the sample (e.g., test strips). In addition, the kit optionally comprises informational material.

In some embodiments, the compositions in the kit can be provided in a watertight or gas tight container which in some embodiments is substantially free of other components of the kit. For example, the swab can be supplied in at least one container (e.g., the container tube), and the sample transport media can be supplied in a container having sufficient reagent for a predetermined number of samples, e.g., 1, 2, 3 or greater. It is preferred that the components described herein are substantially pure and/or sterile.

The informational material can be descriptive, instructional, marketing or other material that relates to the methods described herein. The informational material of the kits is not limited in its form. In one embodiment, the informational material can include information about production of any of the components (e.g., swabs, container tubes, sample transport media), concentration, date of expiration, batch or production site information, and so forth. In one embodiment, the informational material relates to methods for collecting samples using the components of the kit.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Methods of Manufacture and Use

In some embodiments, the swab is manufactured using injection molding, stamping, die cutting, thermal, ultrasonic welding, or 3D printing. In some embodiments, the swab is injection molded. Accordingly, in one aspect described herein is method of manufacturing a swab comprising: (a) injecting a mold with a liquid form of the swab material(s); and (b) removing the swab from the mold once solidified. In some embodiments, the swab material is polypropylene. In some embodiments, the swab material is liquefied, e.g., at a temperature of about 150° C. In some embodiments, the step of removing the swab from the mold comprises use of ejection pins, e.g., that contact at least one incomplete ring of the sample collection head as described herein. The method of manufacturing the swab further comprises a first step of manufacturing the mold, e.g., according to the swab dimensions as described further herein. In some embodiments, only a portion of the swab is injection molded (e.g., neck and/or head) and the portion is then attached to a separate cap, which can be accomplished using an adhesive, a weld, a heat stake, and/or any other known chemical and physical attachment techniques.

In some embodiments, a swab comprising at least two materials can be accomplished using injection molding (i.e., overmolding). Overmolding is a process wherein a single part is created using two or more different materials in combination. Typically, the first material, sometimes referred to as the substrate, is partially or fully covered by subsequent materials (i.e., overmold materials) during the manufacturing process.

n one aspect, described herein is a method of collecting a sample comprising contacting a sample with a swab as described herein. The term “sample” as used herein denotes a sample taken or isolated from a biological organism, e.g., a blood or plasma sample from a subject. In some embodiments of any of the aspects, the present invention encompasses several examples of a biological sample. In some embodiments of any of the aspects, the biological sample is cells, or tissue, or peripheral blood, or bodily fluid. In some embodiments of any of the aspects, the biological sample comprises cells, mucus, and any microorganisms (e.g., bacteria, viruses, fungi). Exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term sample also includes untreated or pretreated (or pre-processed) biological samples. In some embodiments of any of the aspects, a sample can comprise cells from a subject. In some embodiments, the sample is selected from: nasopharyngeal, oropharyngeal, anterior nares, mid-turbinates, any oral surface (e.g., buccal epithelial surface, tongue surface, etc.), and a genital surface (e.g., penis or cervix) of a subject. In some embodiments, the sample is an anterior nare epithelial surface of a subject.

In general, in various embodiments, the swabs described herein can be used to collect any suitable sample to test for infection of any disease state. For example, in some embodiments, the subject is infected with or suspected to be infected with a respiratory infection. In some embodiments of any of the aspects, the respiratory infection is caused by a bacteria, virus, or fungus, e.g., which can replicate in the pulmonary and/or bronchial epithelia. Non-limiting examples of bacteria, virus, or fungi that can cause respiratory infections include: bacteria belonging to one of the Streptococcus, Haemophilus, Staphylococcus, or Moraxella genera (e.g., Streptococcus pneumoniae, Haemophilus influenzae, Staphylococcus aureus, or Moraxella catarrhalis), rhinoviruses (hRV), respiratory syncytial virus (RSV), adenoviruses (AdV), coronavirus (CoV), influenza viruses (IV), para-influenza viruses (PIV), human metapneumovirus (hMPV), or fungi belonging to the Aspergillus genus.

In some embodiments of any of the aspects, the respiratory infection is caused by a coronavirus. The scientific name for coronavirus is Orthocoronavirinae or Coronavirinae. Coronaviruses belong to the family of Coronaviridae, order Nidovirales, and realm Riboviria. They are divided into alphacoronaviruses and betacoronaviruses which infect mammals—and gammacoronaviruses and deltacoronaviruses which primarily infect birds. Non limiting examples of alphacoronaviruses include: Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, and Feline Infectious Peritonitis Virus (FIPV, also referred to as Feline Infectious Hepatitis Virus). Non limiting examples of betacoronaviruses include: Betacoronavirus 1 (e.g., Bovine Coronavirus, Human coronavirus OC43), Human coronavirus HKU1, Murine coronavirus (also known as Mouse hepatitis virus (MHV)), Pipistrellus bat coronavirus HKU5, Rousettus bat coronavirus HKU9, Severe acute respiratory syndrome-related coronavirus (e.g., SARS-CoV, SARS-CoV-2), Tylonycteris bat coronavirus HKU4, Middle East respiratory syndrome (MERS)-related coronavirus, and Hedgehog coronavirus 1 (EriCoV). Non limiting examples of gammacoronaviruses include: Beluga whale coronavirus SW1, and Infectious bronchitis virus. Non limiting examples of deltacoronaviruses include: Bulbul coronavirus HKU11, and Porcine coronavirus HKU15.

In some embodiments of any of the aspects, the coronavirus is selected from the group consisting of: severe acute respiratory syndrome-associated coronavirus (SARS-CoV); severe acute respiratory syndrome-associated coronavirus 2 (SARS-CoV-2); Middle East respiratory syndrome-related coronavirus (MERS-CoV); HCoV-NL63; and HCoV-HKu1. In some embodiments of any of the aspects, the coronavirus is severe acute respiratory syndrome coronavirus 2 (SARS-CoV-2), which causes coronavirus disease of 2019 (COVID19 or simply COVID). In some embodiments of any of the aspects, the coronavirus is severe acute respiratory syndrome coronavirus (SARS-CoV or SARS-CoV-1), which causes SARS. In some embodiments of any of the aspects, the coronavirus is Middle East respiratory syndrome-related coronavirus (MERS-CoV), which causes MERS.

In some embodiments, the subject is infected with or suspected to be infected with a sexually transmitted disease (STD). A few example STDs include: chlamydia, genital herpes, genital warts or human papillomavirus, gonorrhea, hepatitis A, hepatitis B, hepatitis C, syphilis, trichomoniasis, human immunodeficiency virus (HIV), cytomegalovirus, molluscum contagiosum, Mycoplasma genitalium, bacterial vaginosis, scabies, and pubic lice, among many others.

In some embodiments, the subject is infected with or suspected to be infected with an infection detectible through an oral swab. A few examples include: strep throat, pneumonia, tonsillitis, whooping cough, and meningitis, among many others.

In some embodiments, the swab can be used to perform DNA testing (e.g., genomic DNA testing) on the subject.

In some embodiments after the contacting step, the swab is deposited into a container tube. In some embodiments, the container tube contains sample transport media. In some embodiments, the container tube does not contain sample transport media. In some embodiments after the contacting step, the swab or at least a portion of the swab (e.g., the soluble portion) is dissolved, e.g., with water or an aqueous solution if the swab material is water-soluble. Such a dissolving step can permit faster release of the sample from the swab for downstream applications. In some embodiments, after the swab is deposited into a container tube, the sample is processed using a manual process, a semi-automated process, or a fully automated process. In some embodiments, after the swab is deposited into a container tube, the sample is processed using at least one automated device. In some embodiments, the automated device is selected from the group consisting of: a tube capper and decapper machine, a liquid handling machine, and a shaker.

Accordingly, in one aspect described herein is an automated method of processing a swab comprising: (a) receiving a swab as described herein, wherein the swab has been contacted with a sample and deposited into a container tube; (b) removing at least a portion of the sample from the sample collection head using a tube capper and decapper machine, a liquid handling machine, and a shaker; and (c) processing the at least a portion of the sample using a downstream application. In some embodiments, after receiving the swab, a barcode and/or label on the swab and/or collection tube is detected using a barcode scanning machine. In one aspect, described herein is a system capable of performing the methods described herein. For example, the system can include one or more of the following components: a device for removing a cap from a vial, a device for removing a sample from the vial (e.g., by removing a liquid within the vial and/or by removing the swab), a device for transporting the sample to a testing location, a device for testing the sample (e.g., to determine the presence of some substance), a device for controlling one or more of the other devices and/or capturing data resulting from the test conducted on the sample. In some embodiments, the system can perform one or more of the following steps: receiving a swab that has been contacted with a sample and deposited into a container tube, removing at least a portion of the sample from the sample collection head (e.g., using a tube capper and decapper machine, a liquid handling machine, and/or a shaker), and/or processing the at least a portion of the sample using a downstream application. In some embodiments, the system can remove the sample by performing one or more of the following steps: removing the swab from the sample collection tube (e.g., using the tube capper and decapper machine), adding a solution to the sample collection tube (e.g., using a liquid handling machine), replacing the swab into the sample collection tube (e.g., using the tube capper and decapper machine, shaking the solution in the tube in a shaker in order to remove at least a portion of the sample from the sample collection head of the swab, removing the swab from the sample collection tube and solution (e.g., using the tube capper and decapper machine, and/or removing a portion of the solution from the sample collection tube (e.g., using the liquid handling machine) for the downstream application.

In some embodiments, removing at least a portion of the sample from the sample collection head comprises: (a) removing the swab from the sample collection tube using the tube capper and decapper machine; (b) adding a solution to the sample collection tube using the liquid handling machine; (c) replacing the swab into the sample collection tube using the tube capper and decapper machine; (d) shaking the solution in the tube in a shaker in order to remove at least a portion of the sample from the sample collection head of the swab; (e) removing the swab from the sample collection tube and solution using the tube capper and decapper machine; and (f) removing a portion of the solution from the sample collection tube using the liquid handling machine for the downstream application. In some embodiments, the solution is saline.

In some embodiments, the step of removing at least a portion of the sample from the sample collection head is conducted in about 6 minutes. In some embodiments, the step of removing at least a portion of the sample from the sample collection head is conducted in at most 5 minutes, at most 6 minutes, at most 7 minutes, at most 8 minutes, at most 9 minutes, or at most 10 minutes.

In some embodiments, the swab does not inhibit or reduce a downstream application. In some embodiments, the downstream application comprises nucleic acid (e.g., RNA or DNA) extraction, protein extraction, nucleic acid (e.g., RNA or DNA) amplification (e.g., PCR or isothermal amplification methods) and/or a detection assay (e.g., RT-qPCR). Non-limiting examples of isothermal amplification methods include: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), and polymerase Spiral Reaction (PSR). In some embodiments, the downstream application is a diagnostic test, e.g., detection of nucleic acid or protein from at least one microbe of interest. In some embodiments, the downstream application is an automated diagnostic test. In some embodiments, the downstream application comprises a nucleic acid extraction step. In some embodiments, the downstream application comprises RT-qPCR.

In some embodiments, after a soluble swab is dissolved in a buffer for a downstream application, the dissolved swab material (e.g., PVA) represents at most 22% (w/v) of the buffer. In some embodiments, the dissolved swab material (e.g., PVA) represents at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 21%, at most 22%, at most 23%, at most 24%, at most 25%, at most 26%, at most 27%, at most 28%, at most 29%, at most 30%, at most 31%, at most 32%, at most 33%, at most 34%, at most 35%, at most 36%, at most 37%, at most 38%, at most 39%, at most 40%, at most 41%, at most 42%, at most 43%, at most 44%, at most 45%, at most 46%, at most 47%, at most 48%, at most 49%, or at most 50% (w/v) of the buffer.

In some embodiments, the swab (e.g., a dissolved swab) reduces a downstream application(s) by at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%, at most 21%, at most 22%, at most 23%, at most 24%, at most 25%, at most 26%, at most 27%, at most 28%, at most 29%, at most 30%, at most 31%, at most 32%, at most 33%, at most 34%, at most 35%, at most 36%, at most 37%, at most 38%, at most 39%, at most 40%, at most 41%, at most 42%, at most 43%, at most 44%, at most 45%, at most 46%, at most 47%, at most 48%, at most 49%, or at most 50% compared to a downstream application without the swab.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of respiratory infections. A subject can be male or female.

A subject can be one who has been previously diagnosed with or identified as suffering from or having a respiratory infection or one or more complications related to such a respiratory infection, and optionally, have already undergone treatment for a respiratory infection or the one or more complications related to a respiratory infection. Alternatively, a subject can also be one who has not been previously diagnosed as having a respiratory infection or one or more complications related to a respiratory infection. For example, a subject can be one who exhibits one or more risk factors for a respiratory infection or one or more complications related to a respiratory infection or a subject who does not exhibit risk factors.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:

1. A swab comprising a cap, a neck, and a sample collection head formed from a non-flocked material.
2. The swab of paragraph 1, further comprising a threaded portion.
3. The swab of paragraph 2, wherein the cap is removably coupled to the threaded portion, the neck, the sample collection head, or any combination thereof.
4. The swab of any one of paragraphs 1-3, wherein the cap comprises a hollow cylinder with at least one internal groove or at least one internal ridge.
5. The swab of any one of paragraphs 1-4, wherein the cap can interface with an automated device.
6. The swab of any one of paragraphs 1-5, wherein the automated device is a tube capper and decapper machine.
7. The swab of any one of paragraphs 2-6, wherein the threaded portion of the swab is configured to interface with a container tube.
8. The swab of paragraph 7, wherein the threaded portion of the swab is configured to interface with a threaded portion of the container tube.
9. The swab any one of paragraphs 1-8, wherein the head comprises a plurality of spaced apart annular rings, a spiral axis groove, a bulb, a stippled surface, a roughened surface, a textured surface, or any combination thereof.
10. The swab of any one of paragraphs 1-9, wherein the swab is injection molded.
11. The swab of any one of paragraphs 2-10, wherein the threaded portion, the neck, and the sample collection head are fabricated as a unitary item via injection molding, and wherein the unitary item is then adhered to the cap.
12. The swab of any one of paragraphs 1-11, wherein the cap is aligned off-axis relative to the sample collection head.
13. The swab of any one of paragraphs 1-12, wherein the sample collection head is aligned on a first axis, and the cap is aligned on a second axis, the first axis and the second axis being two distinct axes.
14. The swab of paragraph 13, wherein the first axis and the second axis are parallel to each other and spaced apart from each other.
15. The swab of paragraph 13 or 14, wherein the first axis and the second axis are not coaxial.
16. The swab of any one of paragraphs 13-15, wherein the neck is aligned on the first axis with the sample collection head, and wherein the threaded portion is aligned on the second axis with the cap.
17. The swab of any one of paragraphs 1-16, wherein the cap is configured to be grasped by a user.
18. The swab of any one of paragraphs 1-17, further comprising a handle portion coupled to the cap.
19. The swab of paragraph 18, wherein the handle portion extends away from the cap such that the cap is positioned between the handle portion and the neck.
20. The swab of paragraph 18 or paragraph 19, wherein a width of a distal end of the handle portion adjacent to the cap is generally equal to a width of the cap.
21. The swab of any one of paragraphs 18-20, wherein the handle portion has a tapered shape, the handle portion including a distal end having a first diameter and a proximal end having a second diameter.
22. The swab of paragraph 21, wherein the first diameter is less than the second diameter.
23. The swab of any one of paragraphs 18-22, wherein the handle portion is removably coupled to the cap.
24. The swab of paragraph 23, wherein the handle portion is configured to detach from the cap in response to the cap being coupled to a container tube.
25. The swab of paragraph 24, wherein the handle portion is configured to detach from the cap in response to the cap being coupled to the container tube with a correct amount of force or tightness.
26. The swab of paragraph 24 or paragraph 25, wherein the detaching of the handle portion indicates that the cap is sufficiently coupled to the container tube.
27. The swab of any one of paragraphs 23-26, wherein the handle portion is configured to detach from cap in response to application of an external force.
28. The swab of any one of paragraphs 18-27, further comprising a guard positioned at an end of the handle portion adjacent to the cap.
29. The swab of paragraph 28, wherein the guard has a circular shape extending in a plane, and wherein the handle portion extends normal to a plane of the guard.
30. The swab of any one of paragraphs 2-29, wherein the cap, the threaded portion, the neck, and the sample collection head comprise the same material.
31. The swab of paragraph 30, wherein the material is a flexible polymer.
32. The swab of paragraph 30 or 31, wherein the material is polypropylene.
33. The swab of any one of paragraphs 30-32, wherein the material is biodegradable.
34. The swab of any one of paragraphs 30-33, wherein the material is water-soluble.
35. The swab of any one of paragraphs 30-34, wherein the material is hydrophobic.
36. The swab of any one of paragraphs 30-35, wherein the material is polyvinyl alcohol (PVA).
37. The swab of any one of paragraphs 30-36, wherein the material is foam or a porous material.
38. The swab of any one of paragraphs 1-37, wherein the head comprises a fibrous coating.
39. The swab of any one of paragraphs 1-38, wherein the sample collection head comprises a first material, and the remainder of the swab comprises a second material.
40. The swab of any one of paragraphs 1-39, wherein the sample collection head comprises a water-soluble or biodegradable material and the remainder of the swab comprises a flexible polymer.
41. The swab of any one of paragraphs 1-40, wherein the sample collection head comprises PVA and the remainder of the swab comprises polypropylene.
42. The swab of any one of paragraphs 1-41, wherein the neck tapers from a maximum diameter towards the cap to a minimum diameter towards the head.
43. The swab of any one of paragraphs 1-42, wherein the swab has a length that is at most 100 mm.
44. The swab of paragraph 43, wherein the swab has a length that is at most 50 mm.
45. The swab of any one of paragraphs 1-44, in combination with a container tube.
46. A kit comprising the swab of any one of paragraphs 1-45.
47. The kit of paragraph 46, further comprising a container tube.
48. A method of collecting a sample comprising:
contacting a sample with the swab of any one of paragraphs 1-45.
   49. The method of paragraph 48, wherein the sample is an anterior nares epithelial surface of a subject.
   50. The method of paragraph 48 or paragraph 49, wherein the subject is infected with or suspected to be infected with a respiratory infection.
   51. The method of any one of paragraphs 48-50, wherein after the contacting step, the swab is deposited into a container tube.
   52. The method of any one of paragraphs 48-51, wherein after the swab is deposited into a container tube, the sample is processed using at least one automated device.
   53. The method of any one of paragraphs 48-52, wherein the automated device is selected from the group consisting of: a tube capper and decapper machine, a liquid handling machine, and a shaker.
   54. The method of any one of paragraphs 48-53, wherein the swab does not inhibit or reduce a downstream application.
   55. An automated method of processing a swab comprising:
receiving a swab of any one of paragraphs 1-45, wherein the swab has been contacted with a sample and deposited into a container tube;
removing at least a portion of the sample from the sample collection head using a tube capper and decapper machine, a liquid handling machine, and a shaker; and
processing the at least a portion of the sample using a downstream application.
   56. The method of paragraph 55, wherein after receiving the swab, a barcode and/or label on the swab and/or collection tube is detected using a barcode scanning machine.
   57. The method of paragraph 55 or paragraph 56, wherein removing at least a portion of the sample from the sample collection head comprises:
removing the swab from the sample collection tube using the tube capper and decapper machine;
adding a solution to the sample collection tube using the liquid handling machine;
replacing the swab into the sample collection tube using the tube capper and decapper machine;
shaking the solution in the tube in a shaker in order to remove at least a portion of the sample from the sample collection head of the swab;
removing the swab from the sample collection tube and solution using the tube capper and decapper machine; and
removing a portion of the solution from the sample collection tube using the liquid handling machine for the downstream application.
   58. The method of paragraph 57, wherein the solution is saline.
   59. The method of any one of paragraphs 55-58, wherein the step of removing at least a portion of the sample from the sample collection head is conducted in about 6 minutes.
   60. The method of any one of paragraphs 55-59, wherein the downstream application includes a nucleic acid extraction step.
   61. The method of any one of paragraphs 55-60, wherein the downstream application includes RT-qPCR.

### EXAMPLES

### Example 1: Single Shot Injection Molded SBS 96-Well Automation Compatible Anterior Nares Swab

Problem being Solved

One of the key limitations to high throughput diagnostic test, e.g. COVID-19 viral assay, is the time it takes to remove the swab from the sample tube and then transfer the sample to the assay device. This typically involves someone: taking a single sample into a biosafety level 2 (BSL2) space; taking out the swab; transferring the sample; sealing the tube; and then repeating. One institute has 9 full-time equivalents (FTE) to open and process 1500 samples. One university is planning 26 FTEs to process 5000 samples.

The technology described herein replaces this slow manual step with a swab that is automation compatible. With this device a single FTE can remove >1000 swabs in an hour. Because the tube itself can also be barcoded, sample accessioning can easily be automatically linked to sample processing.

### DESCRIPTION OF THE INVENTION

Until recently, the main method of sample collection for respiratory illness was nasopharyngeal (NP) swabs. These swabs are very long and not pleasant for the patient. Recently, anterior nares swabs have been approved for sample collection. Anterior nares (AN) swabs can be much shorter than NP swabs as the swab just only needs to go into the nose to the depth that one can fit their finger.

The swabs as described herein are configured to be used in connection with a cap and a tube that allow for automated processing and handling of the sample. The swabs can include a cap portion that is configured to be coupled to the tube (see e.g., FIG. 1), and the cap can be configured to interface with an automated device used in connection with processing the sample (see e.g., FIG. 2). Described herein is an AN swab that includes a cap or fits in the cap of a tube that is compatible with automation.

FIG. 1 shows a picture of a swab 100 with a sample head 102, a neck 104, a threaded portion 106, and a cap 108. The threaded portion 106 can interface with a corresponding threaded portion of a tube, such that the sample head 102 of the swab 100 is sealed in the tube. The cap 108 can interface with an automated device, so that the automated device can move, control, manipulate, etc. the swab 100 during processing of the sample. The cap 108 of swab 100 includes an internal groove 107 formed by two internally-projecting ridges 109. The groove 107 can aid in allowing the automated device in moving, controlling, manipulating, etc. the swab 100. While only one groove 107 and two ridges 109 are shown, the cap 108 may include any number of grooves 107 and ridges 109.

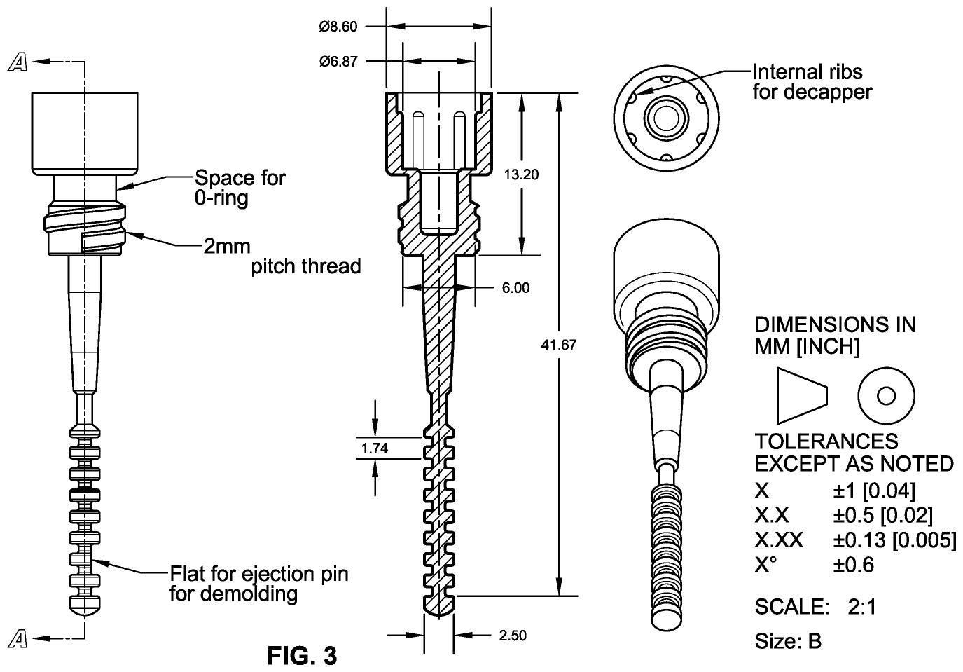

FIGS. 2A, 2B, and 2C shows pictures of the swab 100, how the example swab 100 fits in to a tube 110, and how the swab 100 interfaces with a robotic head 112 of an automated device. As shown in FIG. 2A, the tube 110 may include a barcode or other identifier. As shown in FIGS. 2B and 2C, the tube 110 includes an internally threaded portion 111. When the tube 110 interfaces with the swab 100, the neck 104 fits inside the tube 110, and the threaded portion 106 of the swab 100 interfaces with the threaded portion 111 of the tube 110. In some embodiments, the swab 100 may need to be cut or shortened to fit within tube 110, so that a portion of the swab 100 (such as a portion of the sample head 102) do not fit within the tube 110 when the swab 100 interfaces with the tube 110. In some embodiments, both the sample head 102 and the neck 104 are positioned inside the tube 110 when the threaded portion 106 of the swab 100 interfaces with the threaded portion 111 of the tube 110. Various dimensions of the swab 100 or of other example swabs are shown in FIG. 3.

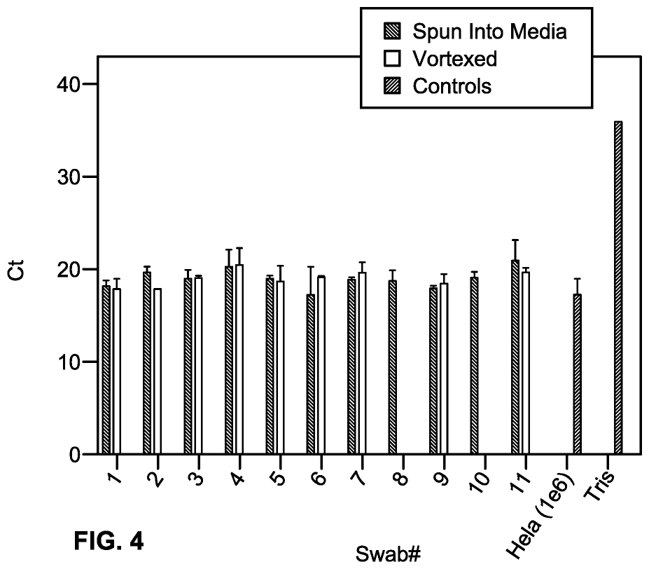

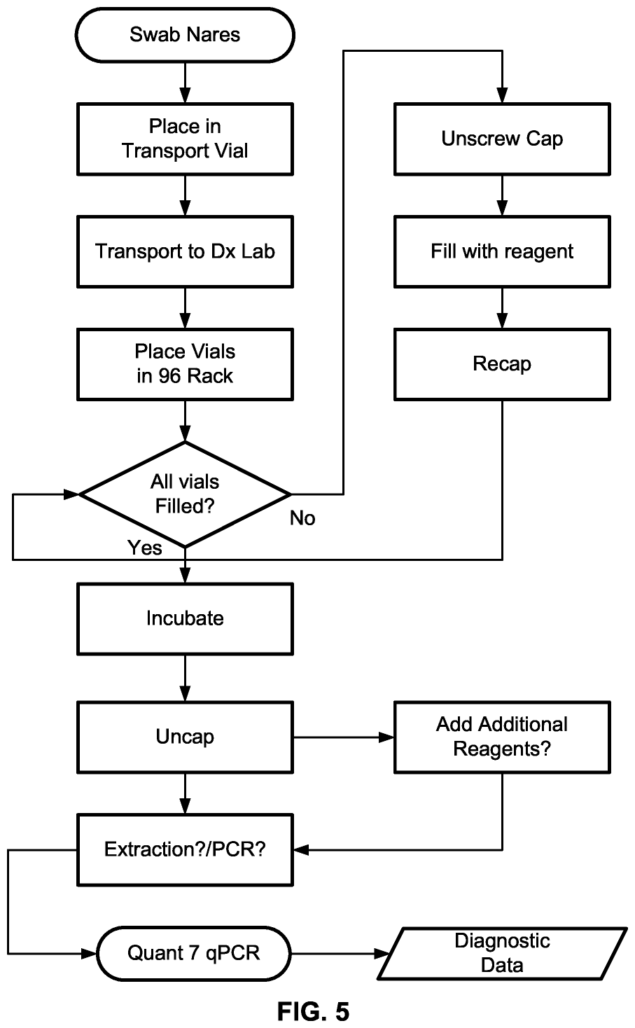

Testing has verified that the swab design is comfortable and is comparable to other swabs for its ability to capture and release RNA and does not inhibit downstream quantitative reverse transcription polymerase chain reaction (RT-qPCR), even without sample extraction (see e.g., FIG. 4). Table 2 below shows the swabs tested in FIG. 4. See e.g., FIG. 5 for an exemplary workflow using cap-integrated swabs.

TABLE 2
Swabs tested in FIG. 4

| Swab # | Swab Description |
|---|---|
| 1 | Microbrush International ™ |
| 2 | PlastCare USA ™ (no bristles) |
| 3 | Swab as described herein |
| 4 | Puritan ™ Sterile Foam Tipped Applicators ™ |
| 5 | Puritan ™ Sterile Polyester tipped applicators ™ |
| 6 | Puritan ™ hydraflock ™ |
| 7 | Super brush ™ 59-1187 |
| 8 | Super brush ™ 59-4582 |
| 9 | BBL ™ Culture swab ™ |
| 10 | BCR ™ Swab Lab Tips ™ |
| 11 | Microbrush International ™ |

### Variations and Optional Features

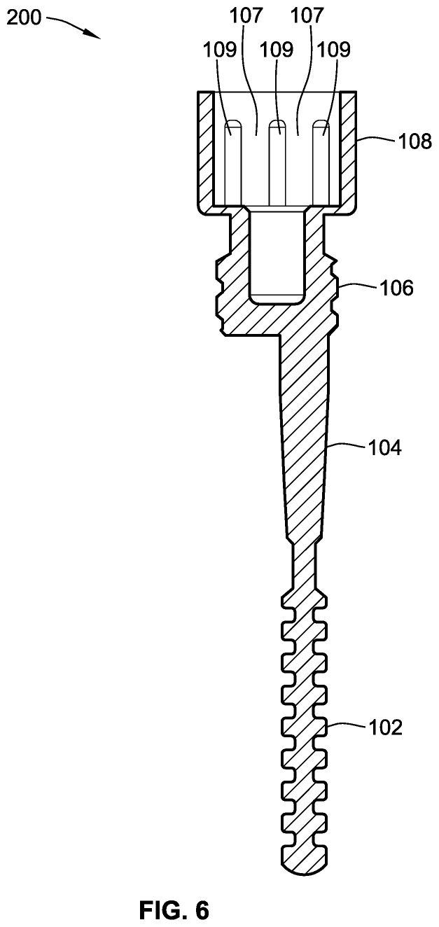

FIG. 6 shows a cross-section of an example swab 200. Swab 200 is similar to swab 100, and includes a sample head 102, a neck 104, a threaded portion 106, and a cap 108. The cap 108 of swab 200 includes two internal grooves 107 formed by three internally-projecting ridges 109. The grooves 107 can aid in allowing the cap 108 of swab 200 to interface with the automated device, similar to swab 100. While only two grooves 107 and three ridges 109 are shown, the cap 108 may include any number of grooves 107 and ridges 109. The sample head 102 and the neck 104 are aligned on a separate axis than the threaded portion 106 and the cap 108. Swab 200 can be used for analyses that utilize an orbital shaker or other actuation that moves liquid in the container tube. The off-axis alignment of the swab 200 results in faster elution toward the walls of the container tube.

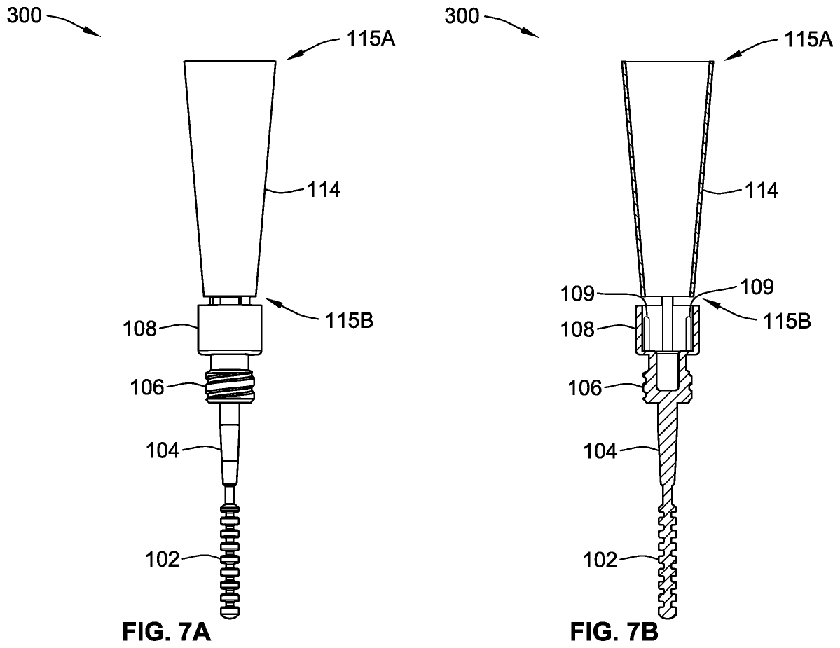

FIG. 7A and FIG. 7B show a perspective view and a cross-section of an example swab 300. Swab 300 is similar to swab 200, and includes a sample head 102, a neck 104, a threaded portion 106, and a cap 108. The cap 108 of swab 300 includes two ridges 109 that form a groove therebetween, to allow the cap 108 of swab 300 to interface with an automated device. While only two ridges 109 are shown, the cap 108 of swab 300 may include any number of ridges 109 (and grooves). The swab 300 further includes a handle portion 114 extending from the cap 108. The user can grasp the handle portion 114, to better control the swab 300 during use. As shown, the handle portion 114 has a tapered shape with a proximal end 115A and a distal end 115B. The width of the proximal end 115A of the handle portion 114 is larger than the width of the distal end 115B of the handle portion 114. In some embodiments, the width of the distal end 115B generally matches the width of the cap 108. In some embodiments, the handle portion 114 has a circular cross-section, and thus the width of the handle portion 114 is the diameter of the handle portion 114.

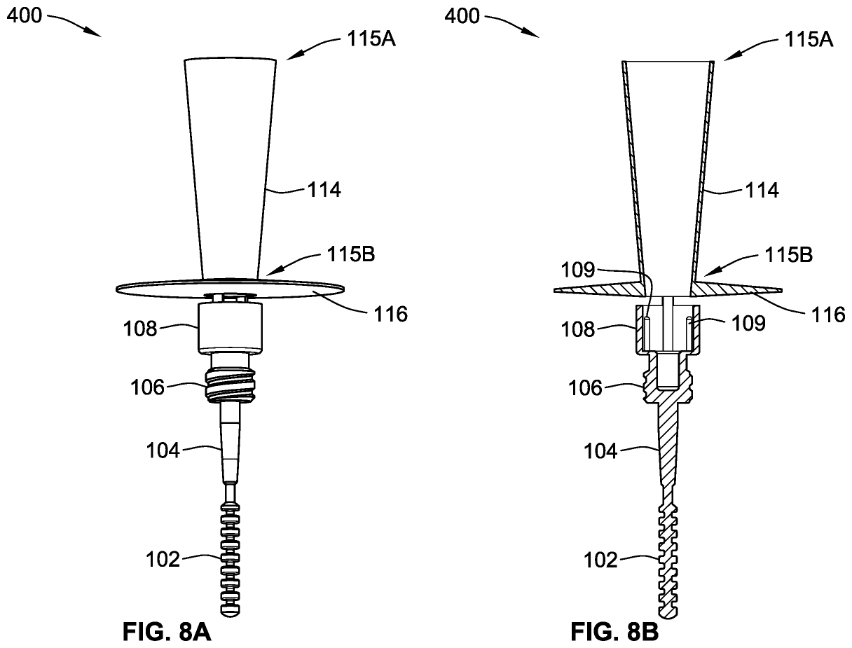

FIG. 8A and FIG. 8B show a perspective view and a cross-section of an example swab 400. Swab 400 is similar to swab 300, and includes a sample head 102, a neck 104, a threaded portion 106, and a cap 108. The cap 108 of swab 400 includes two ridges 109 that form a groove therebetween, to allow the cap 108 of swab 400 to interface with an automated device. While only two ridges 109 are shown, the cap 108 of swab 400 may include any number of ridges 109 (and grooves). The swab 400 further includes a guard 116 located at the distal end 115B of the handle portion 114. The guard 116 aids in preventing the user's fingers from slipping off of the handle portion 114 toward the sample head 102 during use. In some embodiments, the guard 116 has a circular shape, and the handle portion 114 extends in a normal direction relative to the plane of the guard 116.

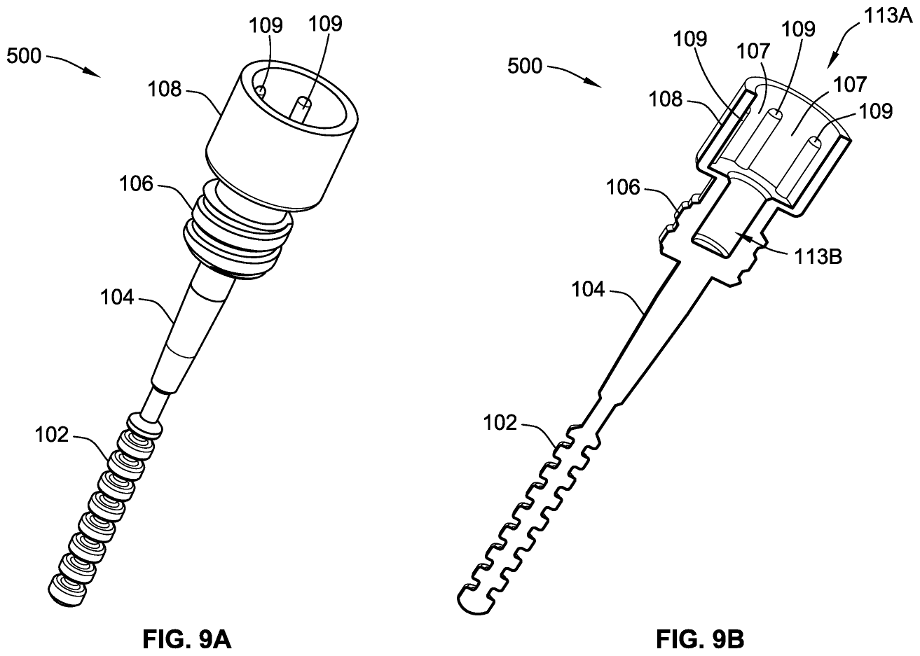

FIG. 9A and FIG. 9B show a perspective view and a cross-section of an example swab 500. Swab 500 can be the same as or similar to any of swabs 100, 200, 300, and 400. Swab 500 includes a sample head 102, a neck 104, a threaded portion 106, and a cap 108. The cap 108 of swab 500 includes three ridges 109 that form two grooves 107 therebetween, to allow the cap 108 of swab 500 to interface with an automated device. While two grooves 107 and three ridges 109 are shown, the cap 108 of swab 500 may include any number of grooves 107 and ridges 109. As shown specifically in FIG. 9B, the cap 108 can include a first internal region 113A where the grooves 107 and the ridges 109 are located. The cap 108 can further include a second internal region 113B. In some implementations, the second internal region 113B has a tapered internal shape, such that the width/diameter of the second internal region 113B decreases toward the distal end. The tapered internal shape of the second internal region 113B can additionally or alternatively aid in allowing the cap 108 to interface with an automated device. In other implementations, the second internal region 113B can have a constant width/diameter, or can even be tapered in the opposite direction, e.g., the width/diameter of the second internal region 113B decreases toward the proximal end.

The basic design of the swab head can be varied with shape or material composition. The cap can be adjusted to fit any standard or custom tube that is compatible with SBS 96-well format. Barcodes or other similar identifiers can be added to the bottom or side of the tube.

Swab head can be flocked or made of an absorbent or soluble material. The swab can be adapted for other standard automation formats. Threads could be designed with different geometry to fit different tube types. Cap can use a snap or bayonet or other attachment type to the tube. Swab can be a different material from the cap.

The swab as described herein comprises at least one of the following features: (1) saves many FTE hours; (2) saves significant space in a Clinical Laboratory Improvement Amendments (CLIA) lab; (3) allows high throughput automation of swab removal; (4) speeds the connection of sample accession to sample processing (e.g., downstream diagnostic applications); (5) single shot injection molded process which allow for cheap and easy manufacturing; (6) head design (e.g., comprising annular rings as described further herein) reduces likelihood of dripping or other cross contamination; (7) compatible with dry or wet transport and self-swabbing at home or at test sites; (8) reduced material consumption due to small size/mass and avoiding need for additional plasticware; (9) cap is used as a handle and prevent risk to patients from over-insertion of swab in the nose; or (10) no need to break swab for collection, which minimizes contamination and infection risk.

### Example 2: Exemplary CLIA Work Flow

### Sample Collection

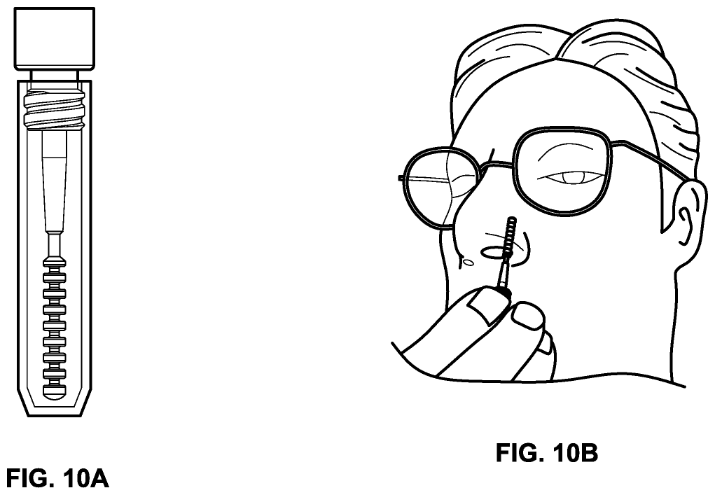

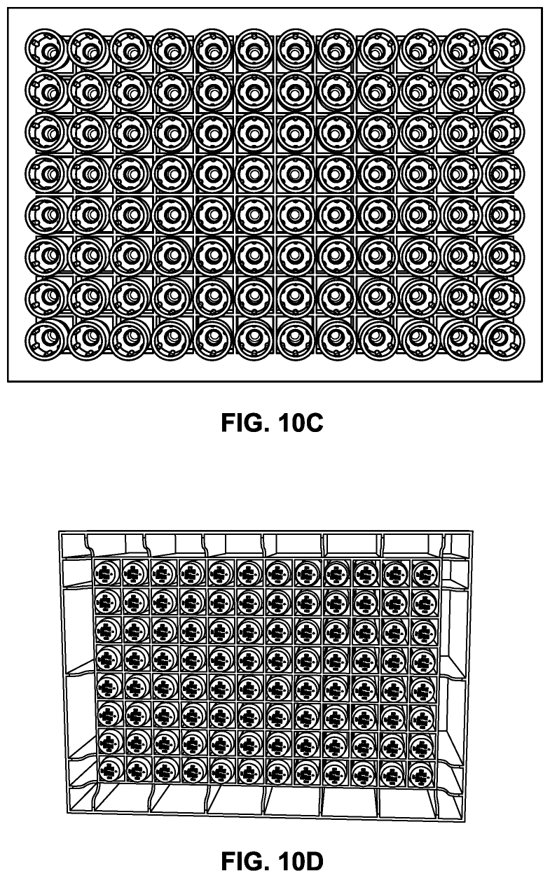

Sample collection can be performed with standard current swabs. However, to the present workflow comprises conducting sample collection with the custom AN swabs described herein (see e.g., FIG. 10A-10B). The swab is a single shot injection molded polypropylene piece, each with a unique barcode on the side and bottom. The swab is unscrewed from the tube and an AN swab is performed by standard methods. These custom AN swabs are produced at scale. There are two options for swab and tube production, which can be pursued simultaneously: (1) parts are ordered directly from a manufacturer using the design from the molds that have already been made, or (2) a partnership with a company to produce the swabs.

Upon receiving the tube, the patient scans the side of the tube (e.g., using a cellphone app, phone-accessed website, or barcode scanner at the collection site). Software for this barcode scanning system has been written and successfully deployed for testing. The associations between patient identity and sample tube barcode are stored at the command center and are not transmitted to the testing facility, ensuring patient anonymity at the testing facility.

After completing the swab, the patient screws the swab back into the tube. In an unsupervised self-collection setting, the tube would be rescanned to ensure no sample switching has occurred. The tubes would be deposited in a lockbox, which would periodically be sent to a testing center. In some embodiments, some or all of the swabs will be stored and transported dry, so there is no risk of liquid leakage. The swabs are stable in this dry form for at least 80 hours. In other embodiments, some or all of the swabs will be store and transported wet. In these embodiments, the swab and/or the container tube can contain an O-ring or gasket to aid in ensuring liquid does not leak from the container tube.

### Sample Accession

In the testing facility, the samples are received and loaded into 96 well racks by hand (see e.g., FIG. 10C). Each rack of tubes is then put onto a robot that scans all 96 barcodes in seconds (see e.g., FIG. 10D). After accessioning, the samples pass to a decapping robot. The robot removes the 96 caps (e.g., 30 seconds) and moves the rack to a liquid handling robot (e.g., 30 seconds), which adds 100 uL of saline solution (e.g., 10 seconds). The liquid handler then moves the rack back to the decapping robot (e.g., 30 seconds), which replaces the caps (e.g., 30 seconds) before moving the tubes to an orbital shaker (e.g., 30 seconds), which shakes to move sample material into solution (e.g., 10 seconds). The racks are then moved to the decapping robot (e.g., 30 seconds), which removes the caps (e.g., 30 seconds), and racks are then moved back to the liquid handler (e.g., 30 second), which moves some volume of sample into a microplate for downstream qPCR (this step has multiple possible workflows depending on how the test will be conducted, see below). Meanwhile, the rack is moved back to the decapper and the caps are put back on and moved to a storage spot (e.g., 1.5 minutes). The total time per 96-well sample tube rack is approximately 6 minutes.

### Workflow 1 (No-Extraction “NoEx” Assay):

In this workflow, the liquid handling robot pipets 1 uL of each sample into a 384-well microplate which has been prefilled with 4 uL of qPCR mastermix (NEB). Once 4 sample tube racks have been quadranted to a 384-well microplate, that microplate is moved to a qPCR machine which conducts the test.

Throughput: 1 decapper and 1 robot with 4 qPCR machines can process 1536 samples in just under 90 minutes. Throughput would be primarily limited by the qPCR machines.

### Workflow 2a (“Standard” Assay):

Alternatively, instead of moving the sample directly to a 384-well plate, the liquid handling robot transfers 200 uL of sample to a 96-well microplate. A standard mag bead extraction is then performed followed by quadranting to a 384-well plate for qPCR. These operations involve multiple steps and take about 20 minutes per 96 well sample tube rack.

Throughput: 1 decapper and 1 liquid handling robot with 1 qPCRs would process 384 samples in just under 90 minutes. The robot would be at capacity. Throughput would be primarily limited by the liquid handling robot.

Workflow 2b (“Standard” Assay) with Pooling:

In this workflow, the liquid handling robot transfers 20 uL of sample to a 96-well microplate, but 10 96-well sample racks are combined together into a single 96-well microplate (total sample volume of 200 uL per well). This pooling operation would take about 5 minutes. A standard mag bead extraction is then performed, followed by quadranting to a qPCR plate, as in workflow 2a; as above this involves multiple steps and takes about 20 minutes per 96 well plate.

Throughput: 1 decapper and 1 liquid handling robot with 1 qPCR machine would process 1536 in 90 minutes. Throughput would be mainly limited by the robot.

qPCR

qPCR can be performed on Quant Studio 7 Flex 384™ qPCR machines using the NEB Luna Universal™ reaction mix. The reaction multiplexes two genes: N1 from SARS-CoV-2 and GAPDH from human (e.g., multi-exon probe). The human gene serves as a process control and helps ensure proper sample collection. Signal can be read out with Taqman™ probes; one fluorescent channel for each gene. The primers for GAPDH can be at lower concentration to ensure they do not saturate the reaction. Each plate contains 2 positive and 2 negative controls.

### Analysis

The qPCR machine returns the cycle time (Ct) at which each gene was detected. Based on the negative controls, a Ct for presence or absence can be established for each gene. The data for each run are processed and results are returned according to Table 3 below, which shows all four possible results.

TABLE 3
Exemplary Results

| BARCODE | N1 | GAPDH | Results | Validity |
|---|---|---|---|---|
| FOIF920FS | + | + | Positive | VALID |
| SW098SDE | + | − | Positive | VALID |
| SOJFW8F8 | − | + | Negative | VALID |
| 8S9SDF8S0 | − | − | Inconclusive | INVALID |

### Communication

Results are then sent back to the command center, which associates results with patients and informs the appropriate parties. The VALID/INVALID status of each test is sent to the test scheduler, which would either acknowledge a proper test or request a repeat test if INVALID. If desired, the system can also schedule a second test for all positive results. The results table would be returned to the health center (and their command center) to initiate the contact tracing process and any decision in changing testing cadence and follow-up with any positive individuals.

Alternative qPCR Testing

The qPCR test as described herein contains two probes (e.g., N1 and GAPDH). The system can work well with up to 4 probes. Two additional probes can be developed (e.g., for influenza A and influenza B; see e.g., the CDC's Diagnostic Multiplex Assay for Flu and COVID-19 and Supplies, available on the worldwide web at cdc.gov/coronavirus/2019-ncov/lab/multiplex.html).

### Example 3: Accessioning and Automation Compatible Anterior Nares Swab Design

The COVID-19 pandemic has resulted in an unparalleled need for viral testing capacity across the world and is a critical requirement for successful re-opening of economies. The logistical barriers to near-universal testing are considerable. Described herein is an injection molded polypropylene anterior nares swab, the RHINOstic™, with a screw cap integrated into the swab handle that is compatible with fully automated sample accessioning and processing. Generally, the RHINOstic™ swab can be the same as or similar to any of swabs 100, 200, 300, 400, and 500 (see e.g., FIG. 1, 2, 3, 6, 7A-7B, 8A-8B, 9A-9B). The ability to collect and release both human and viral material is comparable to that of several commonly used swabs. SARS-CoV-2 is stable on dry RHINOstic™ swabs for at least 3 days, even at 42° C., and elution can be achieved with small volumes. The swab and barcoded tube set can be produced, sterilized, and packaged at <2 USD per unit and can easily be adopted by large research institutes to increase throughput and dramatically reduce the cost of a standard SARS-CoV-2 detection pipeline.

### INTRODUCTION

At least 27 million cases of COVID-19 and 890,000 deaths have been reported world-wide. To determine if a patient has COVID-19, in most cases, a nasopharyngeal (NP) swab is collected by a trained professional. The swab is then deposited in 1-3 mL of transport media followed by RNA purification and RT-qPCR. NP swabs are around 15 cm in length with a collection head coated with short synthetic filaments, flock, or spun fibers; collection is often an uncomfortable process. The high demand for testing during this pandemic has outstripped the supply of NP swabs (and many other critical reagents for testing) resulting in a testing bottleneck. These supply limitations together with a drive towards patient self-collection has spurred the development of alternatives to the standard NP swab. A promising alternative is anterior nares (AN) swabs, commonly referred to as nasal swabs. AN swabs offer a testing sensitivity similar to NP swabs but are easier to use and more comfortable for the patient; see e.g., Irving et al. 2012. Comparison of nasal and nasopharyngeal swabs for influenza detection in adults. Clin Med Res 10:215-218).

The choice of swab and collection device can have a major impact on the testing speed in clinical labs. Upon receiving samples, a typical procedure in a testing facility is to first accession the delivered patient samples by scanning the barcoded label to upload relevant patient data into the system, then swabs are manually removed from each collection tube and disposed of. The sample transport media is then processed to purify RNA, which is used as input for RT-qPCR. The initial steps in this procedure are hard to automate, slow, and expose staff to infection. Standard 1D barcoding systems and the manual removal of swabs is time consuming and thus costly. There are machines that can perform the entire procedure from accessioning to results, one tube at a time, e.g. a Cobas® 8800, but this process is slow, 1056 tubes per 8-hour shift, and the machines are expensive.

In an effort to meet the dramatic increase in demand for nasal swabs, several groups have designed and 3D printed new swabs (see e.g., Callahan et al. 2020. Open Development and Clinical Validation of Multiple 3D-Printed Nasopharyngeal Collection Swabs: Rapid Resolution of a Critical COVID-19 Testing Bottleneck. Journal of Clinical Microbiology; Alghounaim et al. 2020. Low-Cost Polyester-Tipped 3-Dimensionally-Printed Nasopharyngeal Swab for the Diagnosis of Severe Acute Respiratory Syndrome-Related Coronavirus 2 (SARS-CoV-2). J Clin Microbiol.). The performance of these swabs is comparable to that of standard swabs; however, they aim to reproduce the existing status quo, rather than to address some of the limitations caused by the standard swab design. An ideal swab would be one that is comfortable for patients to self-administer without sacrificing performance, while also allowing for automated specimen accessioning and processing. Additionally, the swab would be made from non-absorbent material, allowing samples to be diluted into smaller volumes of transport media than those used in the current procedure, rendering the sample more concentrated and allowing for more sensitive detection of viral RNA. Described herein is the RHINOstic™, a swab that: 1) performs as well as existing AN swabs; 2) is compatible with direct input to RT-qPCR for extraction free SARS-CoV-2 detection; and 3) is compatible with a collection system (swab and tube) that permits automated sample accessioning and processing.

### Materials and Methods

Swab design. The swabs were designed in SolidWorks™ (Dassault Systémes™) and manufactured using single-shot rapid injection molding (Protolabs™) from medical grade FHR P5M4R polypropylene (Flint Hills™), a material compatible with autoclaving (e.g., 121° C., 20 min), ethylene oxide, gamma radiation, and e-beam sterilization. The stacked rings of the swab head permit collection of nasal matrix without the need for an absorbent coating. The cap cavity is compatible with automated decapping robot systems using a square profile adapter head, while the 2 mm pitch external threading mates with the interior threading of sample collection tubes from several major manufacturers (e.g. Matrix™, Micronics™, and LVL™). As the swab described herein is useful for the collection of nasal samples for diagnostic tests, it is called the RHINOstic™ swab.

Absorption of liquid by swab. The swabs used in this study were weighed on an analytical balance before and after a 15 second incubation in 1 mL of nuclease free water. Six replicates were measured and results are reported in Table 4.

Anterior nares self-swabbing to compare swab performance. Several swab types were compared for performance in anterior nares (AN) specimen collection: the RHINOstic™ prototype, Proctor & Gamble (P&G) blue prototype, a Wyss Institute™ flocked prototype, Puritan™ hydraflock, Puritan™ foam, Puritan™ polyester, US Cotton™, and Microbrush®. Per CDC guidelines, volunteers were instructed to insert the swab 0.5 inch into a nostril, rotate three times along the membrane of the nose firmly and leave in place for 10 to 15 seconds, remove, and then repeat this procedure on the other nostril with the same swab to collect nasal matrix. The volunteer was then instructed to place the used swab in a dry 1.5 mL microcentrifuge tube and break the handle if necessary so the tube could close for transport. Prior to RT-qPCR reactions all swabs were suspended in 200 uL of nuclease free 1×PBS. All experiments in this study were approved by an Institutional Review Board, and informed written consent was obtained from volunteers.

RT-qPCR. RT-qPCR reactions were prepared to reach a final volume of 10 μL using 8 μL of master mix and 2 μL of sample. The Luna Universal One Step™ RT-qPCR kit (NEB™) was used for all RT-qPCR reactions. The master mix protocol was adjusted to include 0.25 U/μL of RNaseIn Plus™ (Promega™) for every 10 μL reaction. RT-qPCR reactions were run on the QuantStudio 6 Real Time PCR™ system (Thermo Fisher Scientific™) following the manufacturer recommended Luna™ RT-qPCR protocol. For all reactions, melt curves were used to determine if products were specific or non-specific. All non-specific Tₘs, >0.5° C. from the expected melting temperature are presented as having a Ct of 40. All experiments included at least one negative control which was either 1×PBS or water. The sequences of all primers used are listed in Table 5.

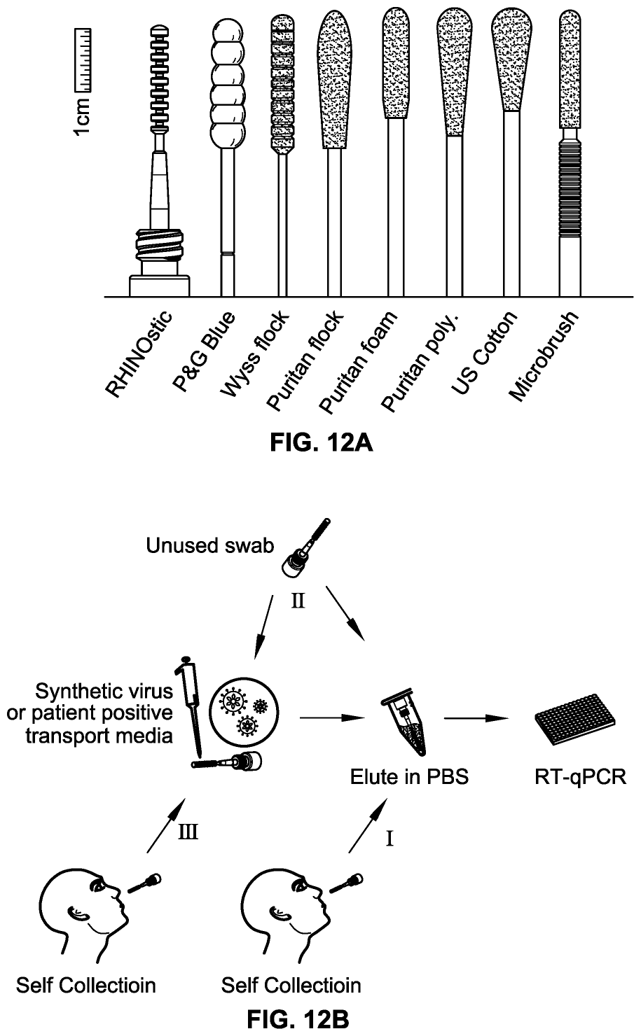

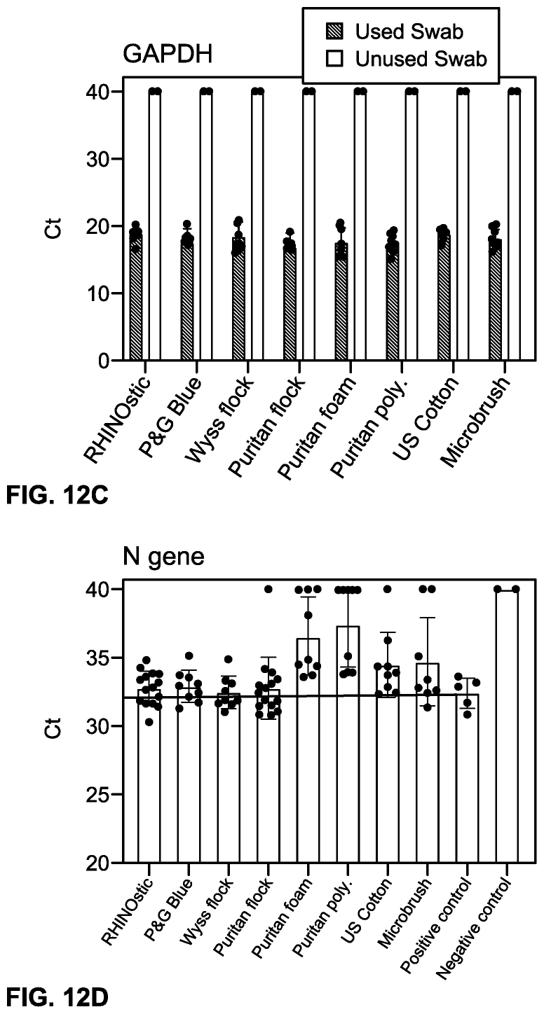

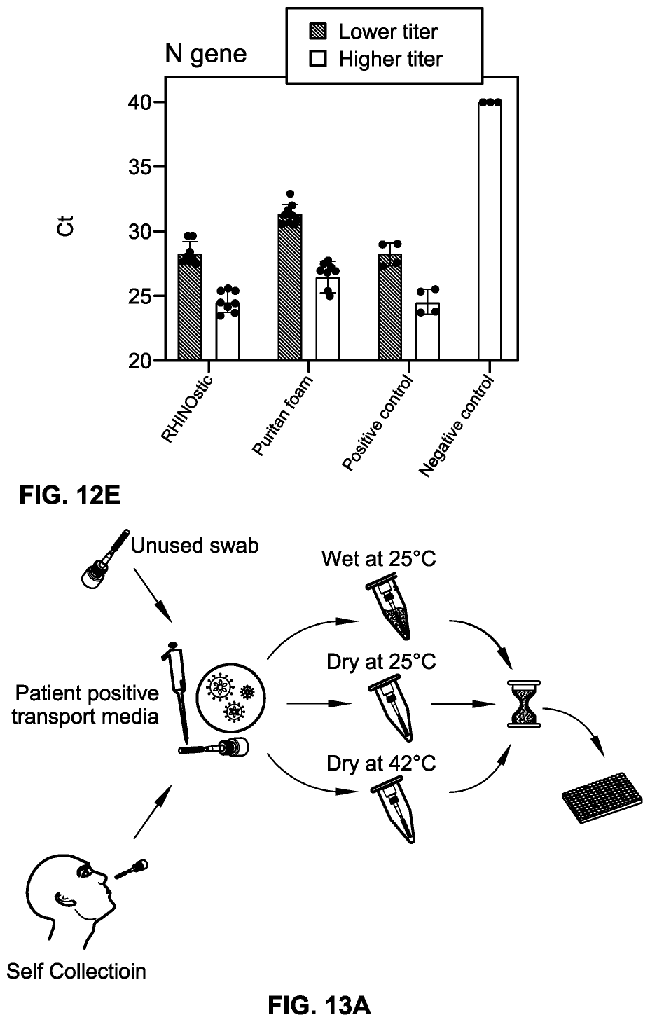

Recovery of human mRNA from AN swabs. SARS-CoV-2 negative volunteers performed AN swabbing as directed with each type of swab tested (see e.g., FIG. 12A-12E, Table 4) to collect nasal matrix. There were three biological replicates for each AN swab measurement, taken on at least two different days. For every condition in which a swab was tested, an unused swab, without nasal matrix, was processed in parallel as a negative control. To recover the sample from the swabs, all swabs were suspended in 200 μL of 1×PBS, vortexed for 10 seconds (sec), spun down in a microcentrifuge, and input directly to the RT-qPCR for GAPDH mRNA detection (see e.g., FIG. 12C).

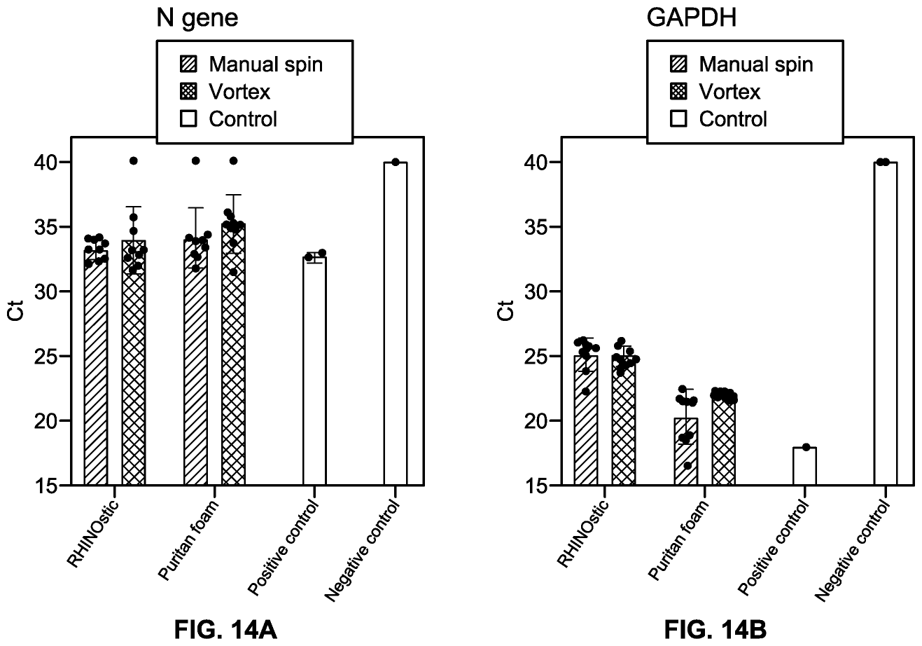

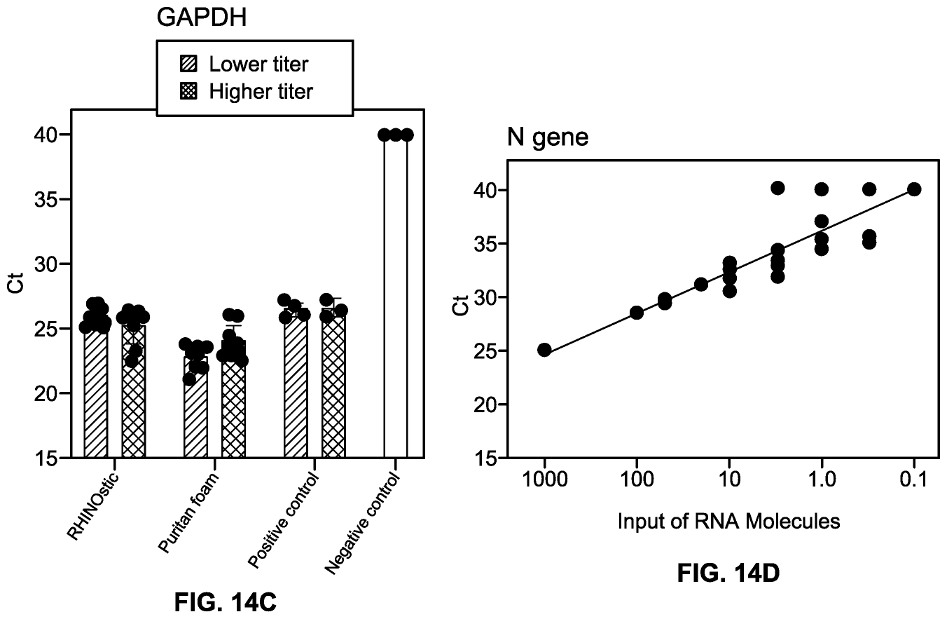

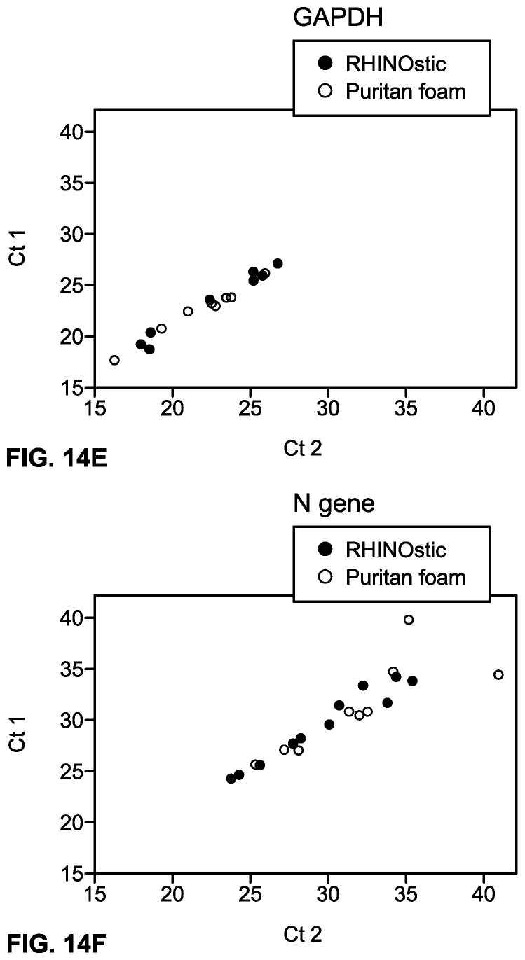

Contrived samples using packaged synthetic SARS-CoV-2 spiked onto unused swabs. AccuPlex™ SARS-CoV-2 verification panel v2 (Seracare™), a packaged synthetic virus, containing the N gene, E gene, ORF1a, S gene, and RdRp was used to simulate the expected viral recovery from AN swabs near the limit of detection (see e.g., FIG. 12D, FIG. 14C). 10 μL of 100 copies/μL packaged synthetic virus was directly applied to the collection head of each swab. Swabs were left in a fume hood for about 20 min until the swabs appeared dry to the eye indicating absorption of the packaged synthetic virus into the collection material. At least three biological replicates were used for every swab tested and replicate data was collected on at least two different days. Swabs were then inserted into a 1.5 mL microcentrifuge tube containing 200 μL of 1×PBS, vortexed for 10 sec, spun down in a microcentrifuge, and 2 μL was input directly to RT-qPCR for N gene detection. The positive control was 10 μL of 100 copies/μL packaged synthetic virus directly input to 190 uL of PBS.

Clinical samples. NP swabs from SARS-CoV-2 patient samples were purchased from BocaBiolistics™, FL. The NP swabs are remnant samples obtained through BocaBiolistics™ and partner labs that were de-identified by BiocaBiolistics™ with their IRB reviewed and approved SOP for de-linking specimens. These NP swabs arrived in 1-3 mL of viral, multitrans, or universal transport media (VTM, MTM, or UTM). 40 μL of each sample was aliquoted and frozen at −80° C. to limit freeze-thawing of samples.

Contrived samples from a clinical source spiked onto swabs with nasal matrix. Nasal matrix was collected from volunteers as described above using RHINOstic™ and Puritan™ foam swabs. 5 μL of clinical sample, with either a higher (1600 copies/μL), or lower titer (˜140 copies/μL), were applied to the collection head of used swabs, and swabs were air dried in the BSL2+ biosafety cabinet for 20 min. Each swab was then placed in a 1.5 mL microcentrifuge tube containing 200 μL of 1×PBS, manually spun for 10 sec in the media, and 2 μL was directly input to the RT-qPCR for both N gene (see e.g., FIG. 12E) and GAPDH mRNA detection (see e.g., FIG. 14C). To assess maximum possible viral recovery from the swab, the positive control was 5 μL of either the higher or lower titer clinical sample in 195 μL of 1×PBS. Negative controls were unused RHINOstic™ and Puritan™ foam swabs suspended in 200 μL of 1×PBS. Three biological replicates were performed for each titer and type of swab tested.

Assessment of stability of SARS-CoV-2 on swabs with nasal matrix over time. To assess the stability of the SARS-CoV-2 virus on swabs with nasal matrix over time, two volunteers self-swabbed three independent times with both the RHINOstic™ and Puritan™ foam swabs for a total of six swabs at each time point. The handles of the Puritan™ foam swabs were broken in order to safely close the collection vial, a 1.5 mL microcentrifuge tube. Several clinical samples were mixed together to generate a pooled clinical sample with a viral titer of ˜10,200 copies/μL. The pooled clinical sample was then aliquoted into 50 μL volumes and refrozen at −80° C. At each time point (72, 48, 24, 2, and 0 hours) an aliquot was thawed and 3 μL of pooled clinical sample was applied to each swab. One RHINOstic™ and one Puritan™ foam swab with nasal matrix from each volunteer was incubated dry at room temperature (25° C.) or 42° C. in a 1.5 mL microcentrifuge tube to assess stability at room temperature or elevated temperatures that may occur during transport. A matched RHINOstic™ or Puritan™ foam swab with nasal matrix from each volunteer was immediately put into a 1.5 mL microcentrifuge tube containing 0.4 mL of 1×PBS to assess the relative stability of a wet swab vs dry swab. An additional 3 μL of the pooled clinical sample was applied to an unused RHINOstic™ and unused Puritan™ foam swab at each time point and kept dry over the time course at 25° C., to assess the effect of nasal matrix on viral recovery. At the end of the time course, dry swabs were suspended in 0.4 mL of 1×PBS. The samples from both wet and dry tubes were mixed by vortexing for 10 sec, then spun down in a microcentrifuge. 2 μL of each sample was directly input to RT-qPCR for GAPDH and N gene detection. The positive control was 3 μL of the pooled clinical sample in 197 μL of 1×PBS at time 0.

### Results

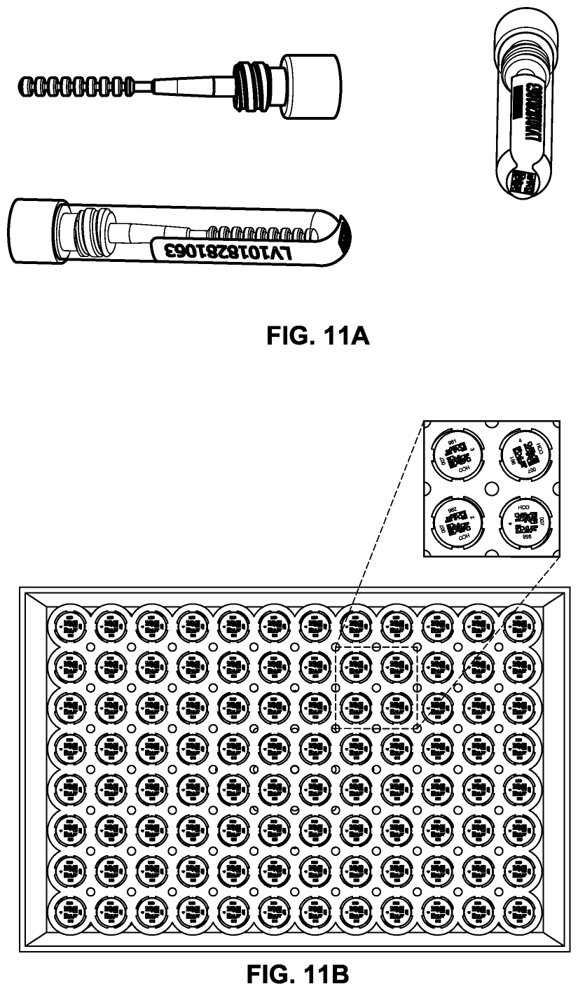

Swab design for automated accessioning and analysis. NP swabs are long, making it challenging to use these swabs with automation-compatible tubes. AN swabs in contrast do not need to be as long as NP swabs and can be designed with a shorter handle, opening up the possibility of making AN swabs that can be directly paired with automation-compatible tubes for an effective collection system. As part of the design, RHINOstic™ swabs have a cap that can be directly screwed onto a 96-well format automation-compatible tube, such as a 1.0 mL Matrix tube (Thermo Fisher Scientific™) (see e.g., FIG. 11A). The swabs were made by single shot injection molding with medical grade polypropylene (see e.g., FIG. 3 and Methods). Injection molding of swabs allows for high volume production at low prices. While the swabs can fit onto many tubes, the optimal design is in collection tubes pre-labeled (e.g., by the manufacturer) with a serialized Type 128 1D barcode plus human readable code on the side with a matching 2D data matrix barcode on the bottom (see e.g., FIG. 11A-11B). This design allows for the collection tube and swab to be accessioned and used by the patient in an unobserved manner without having to pre-register each barcode manually, reducing costs and labor. In addition, the matching 2D barcode on the bottom allows a whole rack of tubes to be accessioned in seconds by a barcode reader.

Swab performance. The RHINOstic™ was compared to several other swabs (see e.g., FIG. 12A, Table 4). First, absorption of water was tested. Water absorption is sometimes used as a proxy for the amount of material that a swab will collect, although it does not necessarily correlate with effective collection of cells and viral particles. The RHINOstic™, as well as the Proctor and Gamble™ (P&G) blue swab absorbed very little water compared to the majority of available swabs (see e.g., Table 4). This lack of absorption is likely because polypropylene is more hydrophobic than the other collection materials, such as cotton and spun polyester.

To test swab performance more directly, the performance of 8 different AN swabs was measured using several approaches (see e.g., FIG. 12A-12E). Collection and recovery was tested of: 1) human mRNA in nasal matrix from swabs, 2) mRNA from viral particles added to swabs, and 3) mRNA from viral particles added to swabs coated in nasal matrix (see e.g., FIG. 12B). Human mRNA was used as a process control to assess successful collection and recovery of cells from swabs. The process control also assesses the efficiency of the reverse transcription (RT) reaction as the primers span two exons to ensure the assay quantifies mRNA not DNA. A single volunteer swabbed with 8 different brands of AN swabs in triplicate (see e.g., FIG. 12B, scheme I) and the eluent was used as direct input for RT-qPCR for GAPDH mRNA to quantify the amount of human mRNA recovered (see e.g., FIG. 12C). All 8 swabs performed similarly in this assay, and no GAPDH was detected on any of the unused swabs (see e.g., FIG. 12C). For all evaluations of AN swabs in this work direct RT-qPCR was performed on the swab eluant without RNA purification.

Recovery of viral particles was first assessed on a contrived sample by applying packaged synthetic SARS-CoV-2 viral particles (Seracare™ reference) to an unused swab for each of the 8 AN swabs tested (see e.g., FIG. 12B, scheme II). The packaged synthetic virus was dried onto the swab and eluted into PBS by vortexing. In a similar experiment, elution into PBS by gentle swirling of the swabs released the virus at equivalent or superior levels to vortexing in the same amount of time (see e.g., FIG. 14A and FIG. 14B). The level of viral particles released by each swab was quantified by RT-qPCR for the SARS-CoV-2 N gene (see e.g., FIG. 12D). The RHINOstic™ performed as well as the other swabs tested, and released an equivalent number of viral particles to the positive control (see e.g., FIG. 12D). The lower detection of viral RNA for other swabs such as the Puritan™ foam is likely due to the fact that these swabs absorb significant volumes of liquid (see e.g., Table 4) making it hard to elute the contents off the swab efficiently, especially given that the maximal recovery of AccuPlex™ possible is 10 molecules per reaction. All subsequent comparisons described below to the RHINOstic™ were performed with only the Puritan™ foam swabs.

To test recovery of SARS-CoV-2 RNA from contrived clinical samples in the presence of nasal matrix, volunteers self-swabbed using either the RHINOstic™ or Puritan™ foam swab, then transport media from SARS-CoV-2 clinical samples was applied to the used swabs (see e.g., FIG. 12B, scheme III). After drying, the viral material was recovered by spinning the swabs in PBS. This experiment was performed with both a lower and a higher titer clinical sample (see e.g., Methods), and the presence of both SARS-CoV-2 N gene and GAPDH mRNA was detected by RT-qPCR using the PBS/swab solution as direct input to RT-qPCR (see e.g., FIG. 12E, FIG. 14C). Additionally, the equivalent performance of the RHINOstic™ to the positive control demonstrates the robustness of RT-qPCR to nasal matrix. The clinical sample titers were determined using an N gene standard curve (see e.g., FIG. 14D, Supplementary Methods). RHINOstic™ swabs were not statistically distinguishable from the positive control at either titer, but the Puritan™ foam swabs showed lower recovery (P<0.001 by an independent t-test). Replicate Ct values shows the high reproducibility of the qPCR data (see e.g., FIG. 14E and FIG. 14F).

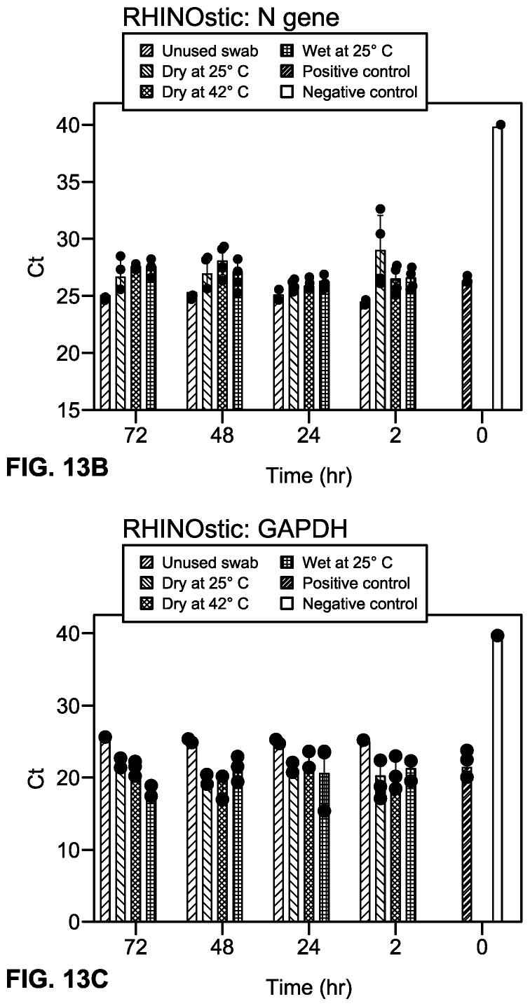

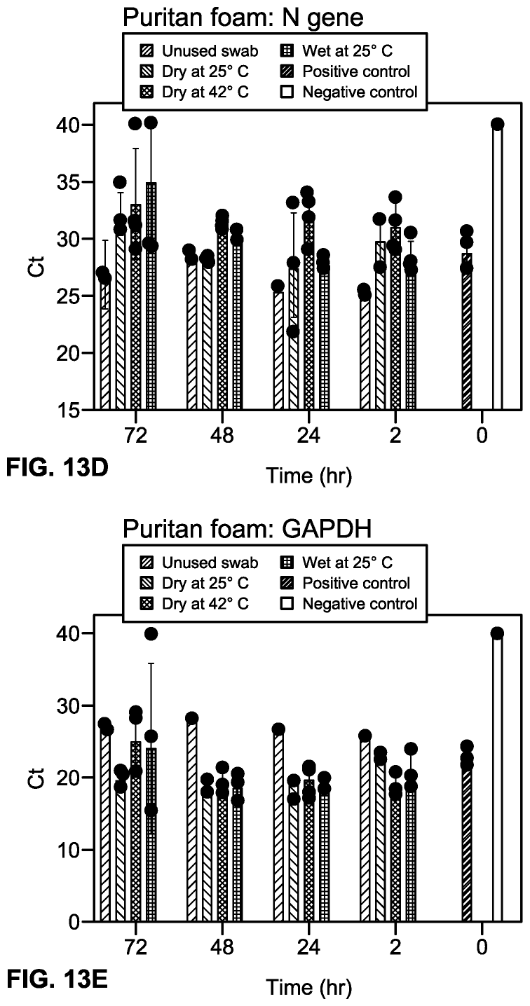

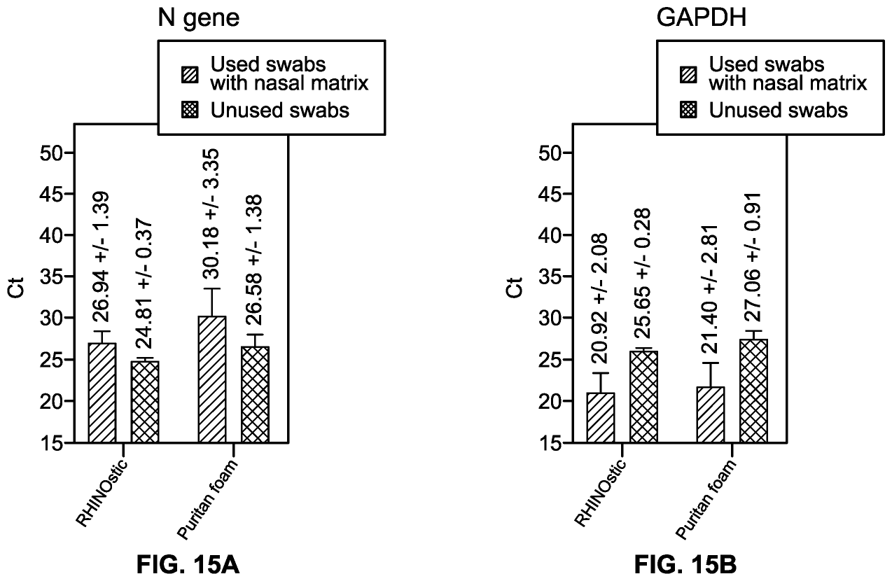

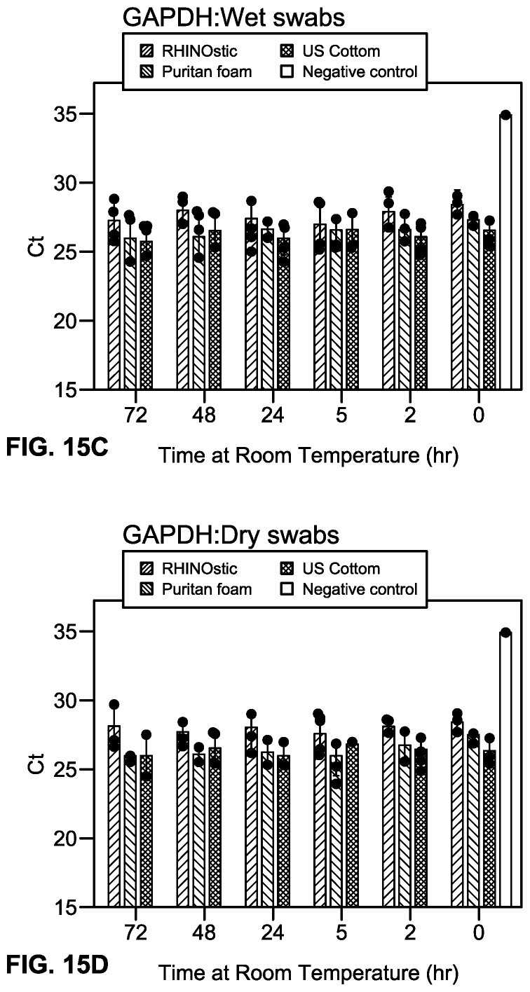

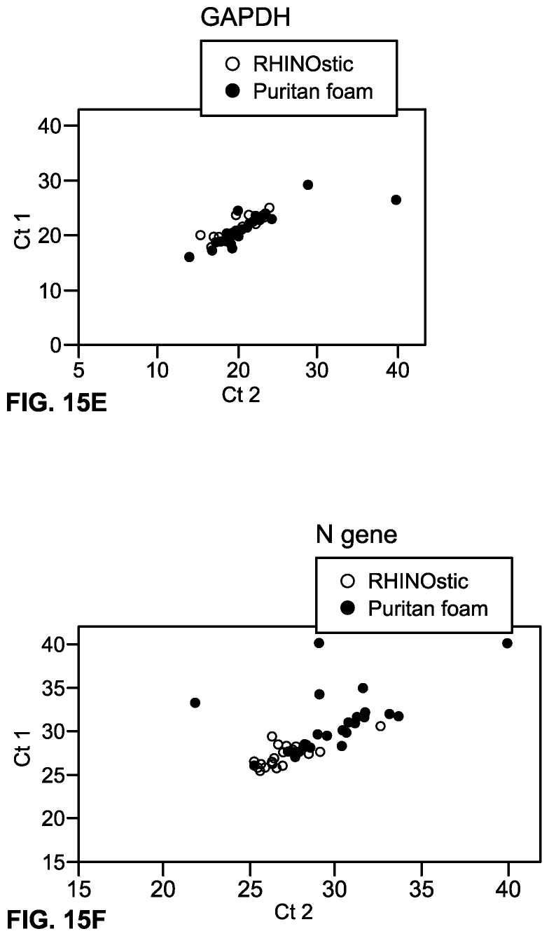

Virus stability on swabs. A key issue with swabs is the stability of viral particles on the swabs during transport from the collection site to the test lab. To test the stability of SARS-CoV-2 on swabs over time, SARS-CoV-2 from clinical samples was added to swabs containing nasal matrix (see e.g., FIG. 13A). The contrived samples were left wet or dry at 25° C. as well as dry at 42° C., to simulate storage in a hot car or truck, for up to 72 hours before elution into PBS. The presence of both SARS-CoV-2 N gene RNA and GAPDH mRNA was detected by using the swab eluent as direct input into RT-qPCR (see e.g., FIG. 13A-13E and FIG. 15A-15F). SARS-CoV-2 viral particles on the RHINOstic™ swabs were stable under all conditions tested both in the presence and absence of nasal matrix (see e.g., FIG. 13B and FIG. 15A) whereas the Puritan™ foam swabs showed much greater variation in N gene detection when in the presence of nasal matrix, particularly when the sample was left out for 72 hours (see e.g., FIG. 13D and FIG. 15A). Overall, GAPDH detection was more consistent for both the RHINOstic™ and Puritan™ foam swabs (see e.g., FIG. 13C and FIG. 13D, FIG. 15B-15D) across all conditions in the time course, but was slightly more variable for the Puritan™ foam swabs stored wet at room temperature for 72 hours (see e.g., FIG. 13E). The variability in the N gene as well as GAPDH data collected from Puritan™ foam swabs during the time course was also observed when comparing the Ct's between two technical replicates in the RT-qPCR data (see e.g., FIG. 15E and FIG. 15F).

### DISCUSSION

The AN swab described herein is comfortable to use, allows patients to perform swabs for themselves, and permits rapid accessioning and processing. The RHINOstic™ performs comparably to currently available swabs, releasing similar amounts of human and viral material into solution after use (see e.g., FIG. 12A-12E). RHINOstic™ and Puritan™ foam swabs detected similar levels of GAPDH mRNA (see e.g., FIG. 13A-13E), while SARS-CoV-2 was detected more consistently from the RHINOstic™ swab with lower titer contrived samples (see e.g., FIG. 12D and FIG. 12E) or after long periods of storage (see e.g., FIG. 13A-13E). All RT-qPCR reactions performed in this study used direct input of swab eluant to the reaction mix without any RNA extraction and as low as 10 molecules could be detected per assay (see e.g., FIG. 12D).

SARS-CoV-2 viral particles on the RHINOstic™ swab proved to be very stable with no statistically significant loss of Ct under all the conditions tested (see e.g., FIG. 13A-13E). One of the key design elements of the RHINOstic™ swab is the ability for a patient to self-collect their AN swab for sample processing. To best use this feature, dry swabs are used in which the swab is put into the collection tube after self-collection in the absence of any buffer. This swab is then be mailed in or collected at a central location without the need for concern over sample leakage in transport. The stability of SARS-CoV-2 on the RHINOstic™ swab for up to 72 hours before processing (see e.g., FIG. 13B and FIG. 13C) demonstrates the feasibility of the dry swab method. An additional advantage of the swab described herein is the ability to elute the sample in a low volume of liquid (e.g., 200 μL), potentially increasing the sensitivity of the direct RT-qPCR method by 5-15 fold compared to standard methods. Most commercial swabs cannot be used with this low elution volume, due to the high volume of liquid absorbed by the swab (see e.g., Table 4).

The RHINOstic™ swabs can be used in the following workflow: the patient scans the side of the barcode on the side of the tube using a cellphone app, phone-accessed website, or scanner and an ID card at the collection site to link the patient and sample together. After swabbing with the RHINOstic™ swab, the patient screws the swab into the barcoded tube. The sample is then packaged for transport. In an unsupervised self-collection setting, the tube can be rescanned at the sample deposition site to help track sample custody. The tubes can be deposited in a lockbox at the site, which are periodically sent to the testing center. All swabs are stored and transported dry avoiding the risk of liquid leakage. In the testing facility, the samples are received and loaded into 96-well racks by hand (see e.g., FIG. 11B). Each rack of tubes is then put onto a robot that scans the 2D matrix codes on the bottom of the tubes thereby linking the sample ID to each plate and plate location in seconds. After accessioning, the samples pass to a de-capping robot, which removes the caps, and the samples can then be eluted, inactivated, and processed for viral quantitation.

As described herein, the RHINOstic™, an injection molded polypropylene swab with a screw cap integrated into the swab handle, performs equal to several commonly used AN swabs at capturing and releasing SARS-CoV-2 viral particles from AN swabs. This AN swab design can expedite SARS-CoV-2 diagnostic testing while significantly reducing costs. These swabs are generally useful for pathogen panel testing at large research institutes.

TABLE 4
Swab Absorption

| | | Average volume | Standard |
|---|---|---|---|
| Swab | Collection Material | absorbed (μL) | deviation (μL) |
| RHINOstic ™ | Polypropylene | 14.4 | 2.2 |
| P&G ™ blue | Polypropylene | 0.7 | 1.0 |
| Wyss ™ flock | Polypropylene and | 65.8 | 3.9 |
| | polyester flock | | |
| Puritan ™ | Polyester flock | 154.1 | 8.9 |
| hydraflock | | | |
| Puritan ™ | Polyurethane foam | 41.3 | 14.4 |
| foam | | | |
| Puritan ™ | Polyester | 155.9 | 9.6 |
| polyester | | | |
| US Cotton ™ | Cotton | 168.8 | 25.4 |
| Microbrush ® | Nylon Flock | 64.9 | 10.1 |

### Supplementary Methods

Elution of viral particles off swabs. 10 μL of 100 mol/μL AccuPlex™ packaged synthetic SARS-CoV-2 virus (Seracare™) was spiked onto six unused RHINOstic™ swabs and six unused Puritan™ foam swabs. Swabs were left to dry in a fume hood for 20 min until the collection heads appeared dry. Swabs were then placed in a 1.5 mL microcentrifuge tube with 200 μL of 1×PBS. Three RHINOstic™ and three Puritan™ foam swabs were vortexed on high for 10 seconds, and the remaining swabs were spun between the index finger and thumb for 10 seconds. 2 μL of each sample was directly input to the RT-qPCR for N gene detection (see e.g., FIG. 14A). The positive control was 10 μL of packaged synthetic virus in 190 μL of 1×PBS, and the negative control was 1×PBS.

Elution of human cells off swabs. A SARS-CoV-2 negative volunteer swabbed as described in Methods with six RHINOstic™ and six Puritan™ foam swabs. All swabs were put into a 1.5 mL microcentrifuge tube with 200 μL of 1×PBS. Cells were released from three RHINOstic™ and three Puritan™ foam swabs by vortexing for 10 seconds on high. The other three RHINOstic™ and three Puritan™ foam swabs were spun between the index finger and thumb to release captured cells. 2 μL of each swab sample was used as input to the RT-qPCR for GAPDH detection in comparison to a positive control which was 1.35e5 mol of total HeLa RNA (see e.g., FIG. 14B).

N-gene Quantitation. Synthetic full genome SARS-CoV-2 RNA (Twist Bioscience™) was serially diluted in nuclease-free water down to 0.005 molecules/μL and 2 μL was used as input to the RT-qPCR for N gene detection (see e.g., FIG. 14D). The standard curve was used to estimate the titers of clinical samples used to generate contrived patient samples (see e.g., FIG. 12E, FIG. 13B-13E).

Stability of human mRNA on swabs over time. At each time point two volunteers swabbed twice with a RHINOstic™ swab, twice with a Puritan™ foam swab, and twice with a US Cotton™ swab. One replicate for each type of swab tested was left dry in a closed 1.5 mL microcentrifuge tube over the time course and the other was left suspended in 1 mL of 1×PBS. All swabs were left at room temperature for 72, 48, 24, 5, 2, and 0 hrs. At time 0 all dry swabs were suspended in 1 mL of 1×PBS. Swabs were all vortexed for 10 sec and then spun down. 2 μL of each sample was input to the RT-qPCR for GAPDH detection (see e.g., FIG. 15C and FIG. 15D).

TABLE 5

| Oligo Name | Alternative Name | Oligo Sequence (5′→3′) | Oligo Target |
|---|---|---|---|
| JQ217 | N-gene Forward | CAACTTCCTCAAGGAACAACATTGC | N gene |
| | | CAAAA (SEQ ID NO: 1) | |
| JQ223 | N-gene Reverse | TGGAGTTGAATTTCTTGAACTGTTG | |
| | | CGACT (SEQ ID NO: 2) | |
| GAPDH_RPAfwd2 | GAPDH Forward | CAAGCTCATTTCCTGGTATGACAAC | GAPDH |
| | | GAATTTG (SEQ ID NO: 3) | |
| GAPDH_RPArev2 | GAPDH Reverse | GGCTGGTGGTCCAGGGGTCTTACTC | |
| | | CTTGG (SEQ ID NO: 4) | |

What is claimed herein is:

1. A swab comprising a cap, a neck, and a sample collection head formed from a non-flocked material.

2. The swab of claim 1, further comprising a threaded portion.

3. The swab of claim 2, wherein the cap is removably coupled to the threaded portion, the neck, the sample collection head, or any combination thereof.

4. The swab of any one of claims 1-3, wherein the cap comprises a hollow cylinder with at least one internal groove or at least one internal ridge.

5. The swab of any one of claims 1-4, wherein the cap can interface with an automated device.

6. The swab of any one of claims 1-5, wherein the automated device is a tube capper and decapper machine.

7. The swab of any one of claims 2-6, wherein the threaded portion of the swab is configured to interface with a container tube.

8. The swab of claim 7, wherein the threaded portion of the swab is configured to interface with a threaded portion of the container tube.

9. The swab any one of claims 1-8, wherein the head comprises a plurality of spaced apart annular rings, a spiral axis groove, a bulb, a stippled surface, a roughened surface, a textured surface, or any combination thereof.

10. The swab of any one of claims 1-9, wherein the swab is injection molded.

11. The swab of any one of claims 2-10, wherein the threaded portion, the neck, and the sample collection head are fabricated as a unitary item via injection molding, and wherein the unitary item is then adhered to the cap.

12. The swab of any one of claims 1-11, wherein the cap is aligned off-axis relative to the sample collection head.

13. The swab of any one of claims 1-12, wherein the sample collection head is aligned on a first axis, and the cap is aligned on a second axis, the first axis and the second axis being two distinct axes.

14. The swab of claim 13, wherein the first axis and the second axis are parallel to each other and spaced apart from each other.

15. The swab of claim 13 or 14, wherein the first axis and the second axis are not coaxial.

16. The swab of any one of claims 13-15, wherein the neck is aligned on the first axis with the sample collection head, and wherein the threaded portion is aligned on the second axis with the cap.

17. The swab of any one of claims 1-16, wherein the cap is configured to be grasped by a user.

18. The swab of any one of claims 1-17, further comprising a handle portion coupled to the cap.

19. The swab of claim 18, wherein the handle portion extends away from the cap such that the cap is positioned between the handle portion and the neck.

20. The swab of claim 18 or claim 19, wherein a width of a distal end of the handle portion adjacent to the cap is generally equal to a width of the cap.

21. The swab of any one of claims 18-20, wherein the handle portion has a tapered shape, the handle portion including a distal end having a first diameter and a proximal end having a second diameter.

22. The swab of claim 21, wherein the first diameter is less than the second diameter.

23. The swab of any one of claims 18-22, wherein the handle portion is removably coupled to the cap.

24. The swab of claim 23, wherein the handle portion is configured to detach from the cap in response to the cap being coupled to a container tube.

25. The swab of claim 24, wherein the handle portion is configured to detach from the cap in response to the cap being coupled to the container tube with a correct amount of force or tightness.

26. The swab of claim 24 or claim 25, wherein the detaching of the handle portion indicates that the cap is sufficiently coupled to the container tube.

27. The swab of any one of claims 23-26, wherein the handle portion is configured to detach from cap in response to application of an external force.

28. The swab of any one of claims 18-27, further comprising a guard positioned at an end of the handle portion adjacent to the cap.

29. The swab of claim 28, wherein the guard has a circular shape extending in a plane, and wherein the handle portion extends normal to a plane of the guard.

30. The swab of any one of claims 2-29, wherein the cap, the threaded portion, the neck, and the sample collection head comprise the same material.

31. The swab of claim 30, wherein the material is a flexible polymer.

32. The swab of claim 30 or 31, wherein the material is polypropylene.

33. The swab of any one of claims 30-32, wherein the material is biodegradable.

34. The swab of any one of claims 30-33, wherein the material is water-soluble.

35. The swab of any one of claims 30-34, wherein the material is hydrophobic.

36. The swab of any one of claims 30-35, wherein the material is polyvinyl alcohol (PVA).

37. The swab of any one of claims 30-36, wherein the material is foam or a porous material.

38. The swab of any one of claims 1-37, wherein the head comprises a fibrous coating.

39. The swab of any one of claims 1-38, wherein the sample collection head comprises a first material, and the remainder of the swab comprises a second material.

40. The swab of any one of claims 1-39, wherein the sample collection head comprises a water-soluble or biodegradable material and the remainder of the swab comprises a flexible polymer.

41. The swab of any one of claims 1-40, wherein the sample collection head comprises PVA and the remainder of the swab comprises polypropylene.

42. The swab of any one of claims 1-41, wherein the neck tapers from a maximum diameter towards the cap to a minimum diameter towards the head.

43. The swab of any one of claims 1-42, wherein the swab has a length that is at most 100 mm.

44. The swab of claim 43, wherein the swab has a length that is at most 50 mm.

45. The swab of any one of claims 1-44, in combination with a container tube.

46. A kit comprising the swab of any one of claims 1-45.

47. The kit of claim 46, further comprising a container tube.

48. A method of collecting a sample comprising:
contacting a sample with the swab of any one of claims 1-45.

49. The method of claim 48, wherein the sample is an anterior nares epithelial surface of a subject.

50. The method of claim 48 or claim 49, wherein the subject is infected with or suspected to be infected with a respiratory infection.

51. The method of any one of claims 48-50, wherein after the contacting step, the swab is deposited into a container tube.

52. The method of any one of claims 48-51, wherein after the swab is deposited into a container tube, the sample is processed using at least one automated device.

53. The method of any one of claims 48-52, wherein the automated device is selected from the group consisting of: a tube capper and decapper machine, a liquid handling machine, and a shaker.

54. The method of any one of claims 48-53, wherein the swab does not inhibit or reduce a downstream application.

55. An automated method of processing a swab comprising:
receiving a swab of any one of claims 1-45, wherein the swab has been contacted with a sample and deposited into a container tube;
removing at least a portion of the sample from the sample collection head using a tube capper and decapper machine, a liquid handling machine, and a shaker; and
processing the at least a portion of the sample using a downstream application.

56. The method of claim 55, wherein after receiving the swab, a barcode and/or label on the swab and/or collection tube is detected using a barcode scanning machine.

57. The method of claim 55 or claim 56, wherein removing at least a portion of the sample from the sample collection head comprises:
removing the swab from the sample collection tube using the tube capper and decapper machine;
adding a solution to the sample collection tube using the liquid handling machine;
replacing the swab into the sample collection tube using the tube capper and decapper machine;
shaking the solution in the tube in a shaker in order to remove at least a portion of the sample from the sample collection head of the swab;
removing the swab from the sample collection tube and solution using the tube capper and decapper machine; and
removing a portion of the solution from the sample collection tube using the liquid handling machine for the downstream application.

58. The method of claim 57, wherein the solution is saline.

59. The method of any one of claims 55-58, wherein the step of removing at least a portion of the sample from the sample collection head is conducted in about 6 minutes.

60. The method of any one of claims 55-59, wherein the downstream application includes a nucleic acid extraction step.

61. The method of any one of claims 55-60, wherein the downstream application includes RT-qPCR.


SEQUENCE LISTING (4 sequences)

SEQ ID NO: 1 - DNA, 30 bp, Artificial Sequence
  synthetic oligonucleotide
caacttcctcaaggaacaacattgccaaaa

SEQ ID NO: 2 - DNA, 30 bp, Artificial Sequence
  synthetic oligonucleotide
tggagttgaatttcttgaactgttgcgact

SEQ ID NO: 3 - DNA, 32 bp, Artificial Sequence
  synthetic oligonucleotide
caagctcatttcctggtatgacaacgaatttg

SEQ ID NO: 4 - DNA, 30 bp, Artificial Sequence
  synthetic oligonucleotide
ggctggtggtccaggggtcttactccttgg